(19) United States
(12) Patent Application Publication
(10) Pub. No.: US 2010/0086919 A1
(43) Pub. Date: Apr. 8, 2010

(54) Devices and methods for immunoglobulin production

(75) Inventor: Frank Daniel McKeon, Strasbourg (FR)

Correspondence Address: ROPES & GRAY LLP, PATENT DOCKETING 39/41, ONE INTERNATIONAL PLACE, BOSTON, MA, 02110-2624, US

(73) Assignee: China Molecular, Ltd., Strasbourg (FR)

(21) Appl. No.: 12/386,175

(22) Filed: Apr. 13, 2009

Related U.S. Application Data
(60) Provisional application No. 61/124,078, filed on Apr. 11, 2008.

(51) Int. Cl. C12Q 1/68; C12Q 1/02; G01N 33/53; C12N 15/01

(52) U.S. Cl. 435/6; 435/29; 435/7.1; 435/7.9; 435/446

(57) ABSTRACT

The present invention relates to devices and methods that are useful for screening and isolating cells that express a desired immunoglobulin. The present invention further relates to devices and methods that are useful in producing monoclonal antibodies from non-immortal cells (such as B cells) or immortalized cells (such as hybridoma cells). The devices and methods disclosed herein significantly improve the efficiency for monoclonal antibody production.

### RELATED APPLICATIONS

This application claims the benefit of the filing date of U.S. provisional application 61/124,078, filed Apr. 11, 2008. The entire teaching of the referenced application is expressly incorporated herein by reference.

### FIELD OF THE INVENTION

The present invention relates to devices and methods that are useful for screening and isolating cells that express a desired immunoglobulin. The present invention further relates to devices and methods that are useful in producing monoclonal antibodies from non-immortal cells (such as B cells) or immortalized cells (such as hybridoma cells).

### BACKGROUND OF THE INVENTION

Antibodies, in particular monoclonal antibodies, have an immense potential as biologics for treating cancer, inflammation, and degenerative diseases (Brekke and Sandlie, Nat. Rev. Drug Discov., 2, 52-62, 2003). Their value derives from the unique elements of the immunoglobulin genes (heavy and light chain loci), which include multiple variable (V), diversity (D), and joining (J) domains. These domains are combined during embryogenesis to generate in excess of 10¹⁴ unique molecules recognizing different antigenic surfaces. Thus, the potential “chemical space” uniquely recognized by antibodies is comparable to that of drug-like small chemical molecules (Lajiness et al., Curr. Opin. Drug Discov. Devel. 7, 470-477, 2004).

However, efforts to exploit the potential uses of monoclonal antibodies have been hindered by their difficulty of production. The standard methods for monoclonal antibody production was first introduced in 1975 (Kohler and Milstein, Nature 256, 495-497, 1975), and have not been significantly altered since then. Briefly, the method involves first immunizing a mouse with an antigen of interest, and then fusing splenocytes from the mouse with murine myeloma cells. The murine myeloma cells are defective in the purine salvage pathway due to a mutation in the HGPRT gene, therefore, fusion cells (hybridomas) can be selected hypoxanthine-aminoterin-thymidine (HAT) medium. Surviving hybridomas are those with a wild type HGPRT gene. These hybridomas are then screened by various approaches to detect those hybridomas that secrete monoclonal antibodies that bind to the original antigen of interest.

A typical good fusion of myeloma cells with splenocytes from an immunized mouse yields about 1000 hybridomas, of which approximately one percent, or five to 10 hybridomas, are positive for a particular antigen. While the standard hybridoma technology has been the basis of monoclonal antibody production over the past two decades, the approach is inherently inefficient and labor intensive. Most importantly, the method only samples a minor fraction of the immunoglobulins available in a typical immune response. To illustrate, a typical fusion involves about 100 million splenocytes, of which approximately one percent, or one million B cells, produce an antibody directed against the immunized antigen. However, the standard fusion process only captures 5 to 10 hybridomas directed at a particular antigen, representing an inefficiency of 50,000- to 100,000-fold. If this efficiency could be significantly improved, monoclonal antibodies could be rendered a powerful rival for small molecules in the medical arena.

Frustration with the inefficiency of hybridoma production protocols has sparked alternative methods for antibody production, such as the phage display approach (Presta, J Allergy Clin Immunol. 116, 731-736, 2005; Dufner et al., Trends Biotechnol. 24, 523-529, 2006).

Under this approach, portions of the immunoglobulin heavy chains containing the complementarity determining regions (CDR1, CDR2, and CDR3) are expressed as part of phage coat proteins. The phages are then selected by affinity for a target antigen. Enhancement of affinity is then performed by mutagenesis and iterative screening. Subsequently, the “positive” sequences are reconstituted into an immunoglobulin heavy chain, and combined with a non-selected immunoglobulin light chain for desired stability. While effective antibodies have been produced by these approaches, the phage display protocols are also complex, labor-intensive, and have limited scaling potential.

The value of monoclonal antibodies as useful tools for diagnostics and immuno-therapies has meant that investigators often have had no alternative but to tolerate long production periods and high development costs. Rapid and efficient ways of producing or screening hybridoma cells that secret a desired antibody are not presently available. Therefore, there is a need for methods and devices to accelerate monoclonal antibody production for experimental, diagnostic and therapeutic applications.

### SUMMARY OF THE INVENTION

The present invention relates to devices and methods that are useful for screening and isolating cells that express a desired immunoglobulin. The present invention further relates to devices and methods that are useful in producing monoclonal antibodies from non-immortal cells (such as B cells) or immortalized cells (such as hybridoma cells). For example, cells expressing a desired immunoglobulin may be screened and selected first, and then immortalized to produce antibody-producing cells (e.g., hybridoma cells). Alternatively, cells expressing a desired immunoglobulin may be selected first and the sequence the immunoglobulin's heavy (e.g., V_{H} region) and/or light (e.g., V_{L} region) chains can be identified. The devices and methods disclosed herein significantly improve the efficiency of monoclonal antibody production.

In one aspect, the invention improves the efficiency of the hybridoma technology by screening and selecting B cells expressing a desired immunoglobulin prior to immortalization (e.g., fusion with immortalized cells such as myeloma cells). Compared with traditional hybridoma protocols, pre-fusion screening can significantly improve fusion efficiency and reduce the labor and costs for hybridomas screening.

The invention takes advantage of the low-level expression of membrane-associated immunoglobulins on B cells to identify and select specific B cell populations in splenocytes or other biological samples (e.g., a blood sample). In certain embodiments, B cells that express a desired immunoglobulin are detected and selected by microbeads (such as magnetic beads). In certain embodiments, B cells that express a desired immunoglobulin detected and selected by florescent or luminescent markers (e.g., FACS). In certain embodiments, microfluidic devices are used to screen and detect B cells that express a desired immunoglobulin prior to immortalization.

In another aspect, the invention provides devices and methods that further improve the efficiency monoclonal antibody production by controlling the immortalization process. In certain embodiments, microfluidic devices are used so that one immortalized cell is fused with one B cell, thereby significantly increasing the number of viable hybridomas.

In one aspect, the invention provides a method for producing an immortalized immunoglobulin-producing cell that expresses an immunoglobulin that binds to an antigen, comprising: a) obtaining a plurality of B cells from a subject, wherein the subject that has been exposed to or immunized with the antigen, or an antigenic portion thereof; b) detecting and selecting from said plurality of B cells one or more B cells that express the desired immunoglobulin as a cell-surface bound immunoglobulin; c) fusing one or more cells of step b) with immortalized cells to produce one or more immortalized immunoglobulin-producing cells.

In certain embodiments, step c) is performed under microfluidic control. In certain embodiments, the immunoglobulin is IgG, IgM, or IgA. In certain embodiments, the immortalize cells are hypoxanthine guanine phosphoribosyl transferase (HGPRT) deficient cell lines, such as myeloma cells, HGPRT-293T cells, etc.

In certain embodiments, one or more B cells that express the desired immunoglobulin is detected and selected by the immunoglobulin's affinity to the antigen, or an antigenic portion thereof.

In certain embodiments, the antigen, or an antigenic portion thereof, is attached to a microbead, such as a magnetic bead. For example, B cells that express the desired immunoglobulin may be detected and isolated by collecting those B cells that bind to magnetic beads coated with the antigen.

In certain embodiments, the antigen, or an antigenic portion thereof, is attached to a detectable marker. In certain embodiments, the detectable marker is a fluorescent marker. In certain embodiments, the detectable marker is a luminescent marker. In certain embodiments, the antigen, or an antigenic portion thereof, is attached to a chromogenic enzyme or an enzyme that generates luminescence (such as beta-galactosidase, alkaline phosphatase, or horseradish peroxidase). For example, B cells that express the desired immunoglobulin may be detected and isolated by fluorescence-activated cell sorter (FACS), e.g., after incubating with a fluorescently-labeled antigen.

In certain embodiments, B cells that express the desired immunoglobulin may be detected and isolated under microfluidic control. For example, a microfluidic device may be used to 1) compartmentalize the B cells into microcapsules, such that only one cell is present in any one microcapsule; 2) detect and select one or more microcapsules in which the compartmentalized cells express the desired immunoglobulin; wherein at least one of the steps 1) or 2) is performed under microfluidic control.

In certain embodiments, after incubation a pool of B cells with an antigen to allow antigen binding, the cells are mixed with a detecting agent, and the detecting agent and the cells are coencapsulated. Alternatively, a detecting agent can also be introduced by separate microcapsules, which are fused with those microcapsules encapsulating the cells.

The invention further provides a method of producing an immortalized immunoglobulin-producing cell, comprising: 1) compartmentalizing immunoglobulin-producing B cells into microcapsules, such that only one B cell is present in any one microcapsule; 2) compartmentalizing immortalized cells into microcapsules, such that only one immortalized cell is present in any one microcapsule; and 3) coalescing a B cell microcapsule formed in step 1) with a microcapsule formed step 2) (e.g., under the influence of an electric field) to cause the fusion of a B cell with an immortalized cell; wherein at least one of the steps 1), 2), or 3) is performed under microfluidic control.

In certain embodiments, the B cells and the immortalized cells are fused by electrofusion techniques. In certain embodiments, the B cells and the immortalized cells are fused by a fusogenic protein. In certain embodiments, the B cells and the immortalized cells are fused using a chemical agent such as polyethylene glycol (PEG).

In another aspect, the invention further provides devices and methods that may be used to remove unsuccessful fusion products and to culture immortalized immunoglobulin-producing cells to form a cell culture.

In certain embodiments, the immortalized cells (such as hybridoma cells) or cell cultures are further screened to detect or confirm the expression of the desired immunoglobulin.

The invention further provides a method for screening immortalized cells (such as hybridoma cells) that express a desired immunoglobulin, comprising: (1) compartmentalizing the immortalized cells (such as a hybridoma cell) into microcapsules, such that only one immortalized cell is present in any one microcapsule; and (2) detecting the presence of the desired immunoglobulin expressed by the compartmentalized cells; wherein at least one of the steps (1) or (2) is performed under microfluidic control.

In certain embodiments, the presence of the desired immunoglobulin is determined by its affinity to the antigen of interest. In certain embodiments, the immunoglobulin-antigen binding is detected by fluorescence. In certain embodiments, the immunoglobulin-antigen binding is detected by luminescence.

In another aspect, the invention provides a method for screening a cell that expresses a desired immunoglobulin among a repertoire of cells, comprising: a) compartmentalizing the repertoire of cells into microcapsules, such that only one cell is present in any one microcapsule; and b) detecting the expression of the desired immunoglobulin by the compartmentalized cells; wherein one of the steps a) or b) is performed under microfluidic control.

In another aspect, the invention provides a method for producing a fusion cell, comprising: a) compartmentalizing a first population of cells into microcapsules, such that only one cell is present in any one microcapsule; b) compartmentalizing a second population of cells into microcapsules, such that only one cell is present in any one microcapsule; and c) coalescing a microcapsule formed in step a) with a microcapsule formed step b) (e.g., under the influence of an electric field) to cause the fusion of a cell from the first population with a cell from the second population; wherein at least one of the steps a), b), or (c) is performed under microfluidic control.

In another aspect, the invention provides a method for producing an immunoglobulin that bind to an antigen, comprising: a) obtaining a plurality of B cells from a subject, wherein the subject that has been exposed to or immunized with the antigen, or an antigenic portion thereof; b) detecting and selecting one or more B cells that express the desired immunoglobulin from the plurality of cells; c) identifying the sequence the immunoglobulin's heavy (e.g., V_{H} region) and/or light (e.g., V_{L} region) chains from the selected B cell(s). In certain embodiments, one or more steps to identify the sequence of the immunoglobulin are performed under microfluidic control. In certain embodiments, the sequence the immunoglobulin is identified by PCR.

In another aspect, the invention provides a method of identifying at least two B cells, each expressing an immunoglobulin that binds to an antigen, comprising: a) obtaining a plurality of B cells from a subject, wherein the subject that has been exposed to or immunized with at least two different antigens, or their antigenic portion thereof; b) attaching each antigen, or its antigenic portion thereof, to a unique detectable marker, such that each of the antigens is associated with a different detectable marker; c) detecting and selecting two or more B cells that express the desired immunoglobulins from the plurality of cells, wherein the binding of an immunoglobulin to an antigen is determined by the unique detectable marker associated with the antigen. In certain embodiments, the method further comprises fusing the selected B cells of step c) with immortalized cells to produce immortalized immunoglobulin-producing cells. In certain embodiments, the method further comprises identifying the sequences the immunoglobulins heavy and/or light chains from the selected B cells of step c).

### BRIEF DESCRIPTION OF THE DRAWINGS

FIG. 1 schematically illustrates an embodiment of a method in accordance with the present invention for producing monoclonal antibodies.

FIG. 2 is a flow chart illustrating four exemplary procedures to select B cells that express a desired antigen prior to immortalization. A. FACS sorting. B. Magnetic bead-based sorting. C. Microfluidic controlled screening of B cells based on luminescent sorting. D. Microfluidic controlled screening of B cells based on fluorescent sorting.

FIG. 3 is a schematic illustrating the interacting modules of an exemplary microfluidic device.

FIG. 4 shows exemplary cell expansion devices that may be used to remove unsuccessful fusion products and to culture hybridoma cells. A. A cross-sectional view of an exemplary cell expansion device. B and C. Exemplary devices and methods of transferring cells from a collection device to an expansion device. D. A cross-sectional view of an exemplary detection device to detect immunoglobulin-producing hybridoma cells.

FIG. 5 is a cross-sectional view of another exemplary cell expansion device.

FIGS. 6A and 6B illustrate several exemplary means of electrofusion by which non-immortalized B cells can be fused with other cells, such as myeloma cells and other cells to create an immortalized cell line for monoclonal antibody production.

FIGS. 7A and 7B show the detection of cell-surface IgG on antigen specific hybridoma cells. NSO: mouse myeloma cells, which do not produce IgG and are therefore commonly used as fusion partners in producing hybridomas with B cells. Clone 1 (also known as 76A6): a hybridoma cell line secreting an antibody against human Ephrin B1. Clone 2 (also known as 9C3): a hybridoma cell line secreting an antibody against Ephrin B1. The graphs show that surface IgG molecules on the hybridoma cell lines were readily detected. Using a fluorescently-labeled antigen, antigen-specific cells were detected and sorted by FACS.

### DETAILED DESCRIPTION OF THE INVENTION

### 1. Overview

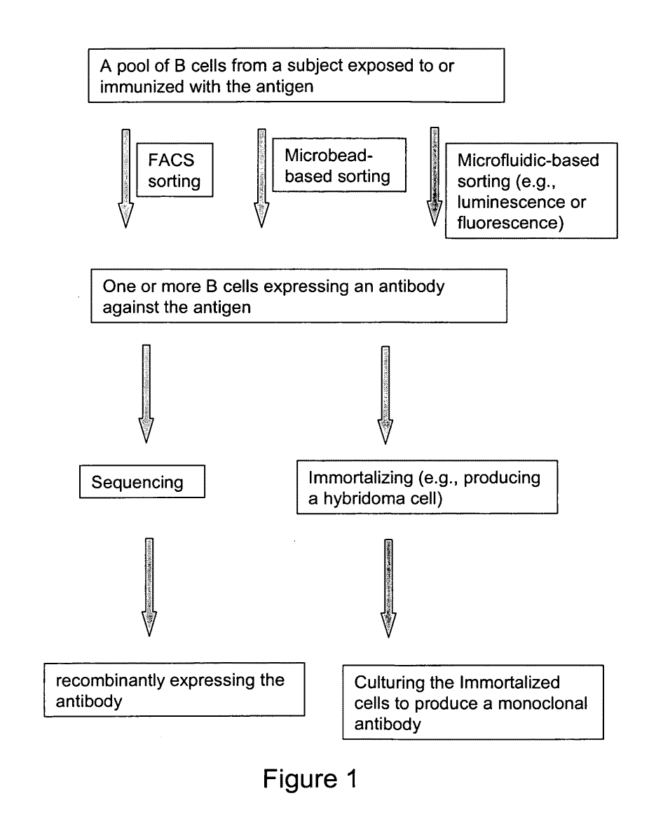

The present invention relates to devices and methods that are useful for screening and isolating cells that express a desired immunoglobulin. The present invention further relates to devices and methods that are useful in producing monoclonal antibodies from non-immortal cells (such as B cells) or immortalized cells (such as hybridoma cells). See, FIG. 1. The devices and methods disclosed herein significantly improve the efficiency for monoclonal antibody production.

One key weakness of the traditional hybridoma techniques originates from the fusion step, whereby typically 100 million splenocytes are fused with 25 million myeloma cells in a mass reaction in the presence of polyethylene glycol (PEG). Several factors contribute to the inefficiencies arising from such a mass reaction. First, many inappropriate fusion products are generated, such as myeloma-myeloma fusions, B cell-B cell fusions, multiple myeloma cell-one B cell fusions, or one myeloma-multiple B cells fusions. These fusion products are likely inviable. Second, successful fusions (one myeloma cell-one B cell fusions) have to be plated and screened for specificity. Sometimes, antibody-producing hybridomas are not detected by the traditional screening methods because of insufficient number of activated B cells-only about 60% of antigens can yield an immune response involving about 1% of B cells; with respect to the remaining 40% or so antigens, the number of activated B cells are considerably lower. Third, because hybridomas grow at different rates, the point at which one must perform the assay for antibody production to assess positive pools of cells can vary and may require more than one assay point on the same pool of cells. During this process, the rapidly growing cells need to be passaged in order to promote viability and to prevent loss of potentially positive clones. Finally, limiting dilution must be performed to achieve clonal populations. Successive rounds of limiting dilutions may be required to achieve clonal or near-clonal populations.

Therefore, the present invention provides novel methods and devices for producing monoclonal antibodies. In certain embodiments, the invention significantly improves the efficiency of the traditional hybridoma technology by screening and selecting B cells expressing a desired immunoglobulin prior to immortalization (e.g., fusion with an immortalized cell, such as a myeloma cell). Compared with traditional protocols, pre-fusion screening can significantly improve fusion efficiency and reduce the labor and costs for hybridoma screening.

The invention takes advantage of the low-level expression of membrane-associated immunoglobulins on B cells to identify and select specific B cell populations in splenocytes or other biological samples (e.g., a blood sample).

In certain embodiments, B cells expressing the desired immunoglobulin may be detected and isolated by collecting those B cells that bind to magnetic beads coated with an antigen, or an antigenic portion thereof.

In certain embodiments, B cells expressing the desired immunoglobulin may be detected and isolated by fluorescence-activated cell sorter (FACS), e.g., after incubating with a fluorescently-labeled antigen, or an antigenic portion thereof.

In certain embodiments, microfluidic devices (such as those available from Raindance Technologies, Lexington, Mass.; www.raindancetechnologies.com) are used to screen and detect B cells that express a desired immunoglobulin prior to immortalization.

In certain embodiments, the invention provides devices and methods that further improve the efficiency monoclonal antibody production by controlling the immortalization process. In certain embodiments, microfluidic devices are used so that one immortalized cell is fused with one B cell, thereby significantly increasing the number of viable hybridomas.

In certain embodiments, after selecting one or more B cells that express the desired immunoglobulin, the sequences the immunoglobulin's heavy (e.g., V_{H} region) and/or light (e.g., V_{L} region) chains from the selected B cell(s) are identified, e.g., by PCR or other techniques known in the art. In certain embodiments, one or more steps to identify the sequence of the immunoglobulin are performed under microfluidic control. For example, single-cell PCR may be used to amplify the mRNA(s) encoding the heavy and light chain sequences of the desired immunoglobulin.

The invention also allows for simultaneous or sequential selection of multiple clones of antigen-producing B cells, each of which expressing an immunoglobulin that binds to a different antigen.

Throughout the specification, the terms “immunoglobulin” and “antibody” are used interchangeably.

As used herein, the term “subject” refers to a warm blooded animal, preferably a mammal, such as a human.

As used herein, the term “antigen” refers to a molecule capable of being bound by an antibody or a T cell receptor (TCR) if presented by MHC molecules. The term also encompasses T-cell epitopes. A T-cell epitope is recognized by a T-cell receptor in the context of a MHC class I, present on all cells of the body except erythrocytes, or class II, present on immune cells and in particular antigen presenting cells. This recognition event leads to activation of T-cells and subsequent effector mechanisms such as proliferation of the T-cells, cytokine secretion, perforin secretion etc. An antigen is capable of being recognized by the immune system and/or being capable of inducing a humoral immune response and/or cellular immune response leading to the activation of B- and/or T-lymphocytes. An antigen can have one or more epitopes (B- and T-epitopes). Antigens include but are not limited to allergens, self antigens, haptens, cancer antigens and infectious disease antigens as well as small organic molecules such as drugs of abuse (like nicotine) and fragments and derivatives thereof. Furthermore, antigens used for the present invention can be peptides, proteins, domains, carbohydrates, alkaloids, lipids or small molecules such as, for example, steroid hormones and fragments and derivatives thereof. The terms “antigen” and “immunogen” are used interchangeably.

### 2. Immunization and Preparation of B Cells

Immunization of a suitable animal and preparation and isolation of B cells may be carried out according to standard techniques. A suitable animal (e.g., rabbit, goat, etc.) may be immunized with an antigen of interest, or an immunogenic portion thereof. Methods for immunizing non-human animals such as mice, rats, sheep, goats, pigs, cattle and horses are well known in the art. See, e.g., Harlow and Lane, Antibodies: A Laboratory Manual, New York: Cold Spring Harbor Press, 1990.

The antigen or immunogen may be a full length protein of interest or an immunogenic peptide derived from the antigen. In some embodiments the immunogen is a peptide of from 7 to 20 amino acids in length, preferably about 8 to 17 amino acids in length. Peptide antigens suitable for producing antibodies of the invention may be designed, constructed and employed in accordance with well-known techniques. See, e.g., Harlow & Lane Eds., Cold Spring Harbor Laboratory (1988); Czernik, Methods In Enzymology, 201: 264-283 (1991); Merrifield, J. Am. Chem. Soc. 85: 21-49 (1962)).

In some embodiments the immunogen is administered with an adjuvant. Suitable adjuvants are well known to those of skill in the art. Exemplary adjuvants include complete or incomplete Freund's adjuvant, RIBI (muramyl dipeptides) or ISCOM (immunostimulating complexes).

In some embodiments, antibody-producing B cells are isolated from an animal immunized with an antigen as described above. Antibody-producing B cells may be isolated from the spleen, lymph nodes or peripheral blood. Individual B cells may be isolated and screened (as described below) to identify cells producing an immunoglobulin specific for the antigen of interest. Identified cells may then be immortalized and cultured to produce a monoclonal antibody according to the teachings of the invention, as well as techniques well known in the art.

In certain embodiments, antibody-producing B cells can be isolated from the blood or other biological samples of a subject suffering from an infection, cancer, an autoimmune condition, or any other diseases to identify a pathogen-, tumor-, and disease-specific antibody of potential clinical significance. For example, the subject may be one that was exposed to and/or who can make useful antibodies against an infectious agent (e.g., viruses, bacteria, parasites, prions, etc). Similarly, some human subjects may produce antibodies against toxic molecules such as drugs of abuse or other toxins, and these antibodies can be isolated using methods and articles described herein. It should be noted that the subject is not necessarily one that appears sick. The subject may be healthy, but produce antibodies of interest. As an example, cancer patients may produce antibodies specific to cancer-cell surface markers. By identifying or determining the antibody-producing cells that produce antibodies against an antigen of interest, such antibodies may be produced, as discussed in detail below, and administered to the subject and/or to other subjects, depending on the application.

### 3. Detecting B Cells that Express Desired Immunoglobulins

The present invention provides novel methods for screening and selecting cells that express a desired immunoglobulin from a mixture of cells, such as spleen cells isolated from immunized animals, or blood or other biological samples from a subject who has been exposed to an antigen of interest. Antibody-producing cells can be screened and selected, e.g., by FACS, by microbeads, or in a microfluidic separation channel or region.

In particular, the invention takes advantage of the low-level expression of membrane-associated immunoglobulins on B cells to identify and select specific B cell populations in splenocytes. In addition to the better-known membrane association of IgM isoforms, each of the constant regions of the IgG immunoglobulins (IgG2A, IgG2B, IgG3) also contain highly conserved pairs of exons (M exons) encoding transmembrane and cytoplasmic domains (Kinoshita et al., Immunol. Lett. 27, 151-155, 1991; Kaisho et al., Science 276, 412-415, 1997). For example, each constant region in the human immunoglobin heavy chain locus (Chromosome 14-5′ m, d, g3, a1, g2, g4, e1, a2) contains two exons, M1 and M2, that encode relatively conserved transmembrane and cytoplasmic domains:

(SEQ ID NO: 1)

```
          transmembrane
WTTITIFITLFLLSVCYSATVTFF
              cytoplasmic
KVKWIFSSVVDLKQTIIPDYRNMIGQGA
```

These membrane-associated IgGs form a so-called B cell receptor (BCR) with CD79 molecule, and the signaling of this complex is essential for the generation of the respective secreted isoforms (Tashita et al., J. Clin. Invest. 101, 677-681, 1998; Terada et al., Int. Immunol. 13, 249-256, 2001).

Detecting these cell surface immunoglobulins can be done in any number of ways, such as enzymatic or fluorescence analysis of antigen binding to the surface of B cells, or a microbead-based assay.

(A) Fluorescence-Activated Cell Sorting (FACS)

In one embodiment, cells expressing a desired immunoglobulin are screened and sorted using fluorescence-activated cell sorting (FACS). FACS is a powerful system which not only quantifies the fluorescent signal but also separates the cells that contain preselected characteristics (such as fluorescence intensity, size and viability) from a mixed population. Laser light is directed at individual cells as they flow through the FACS. A light scatter pattern is generated when the dense nuclear material of the cell interferes with the path of the laser beam. Thus, cells can be selected at random based on their ability to scatter laser light. FACS sorting with multi-color analyses is particularly convenient.

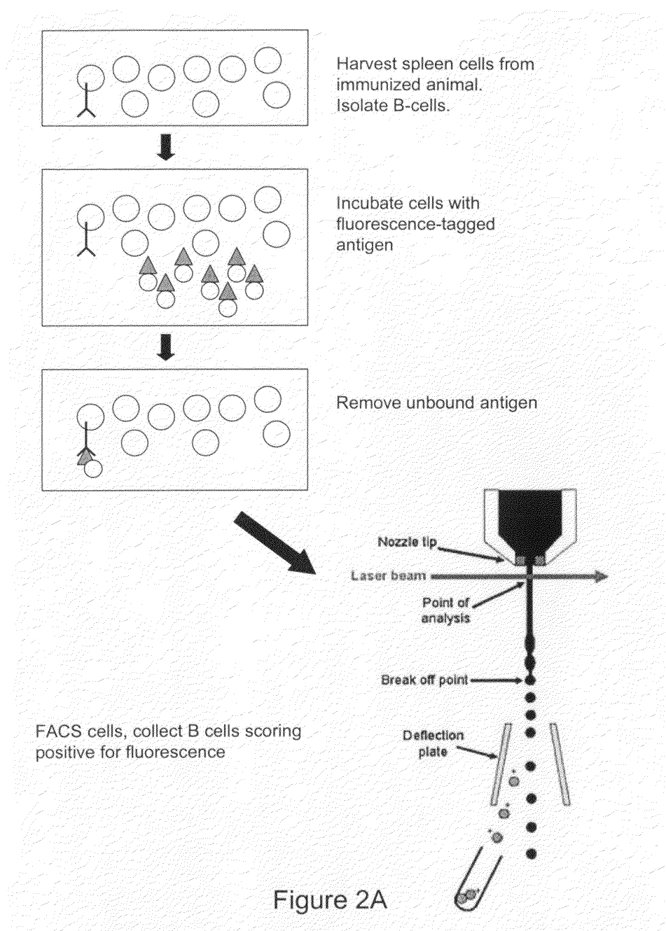

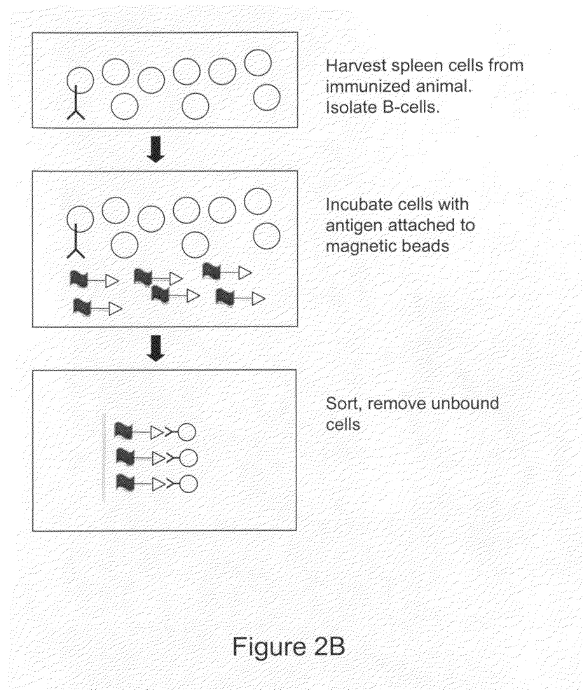

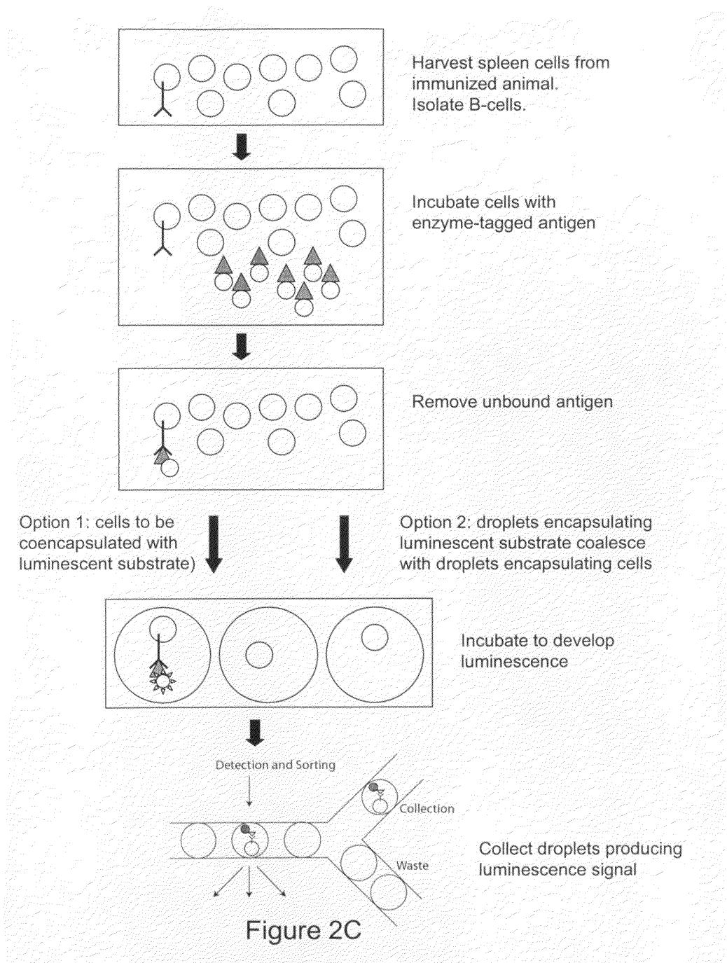

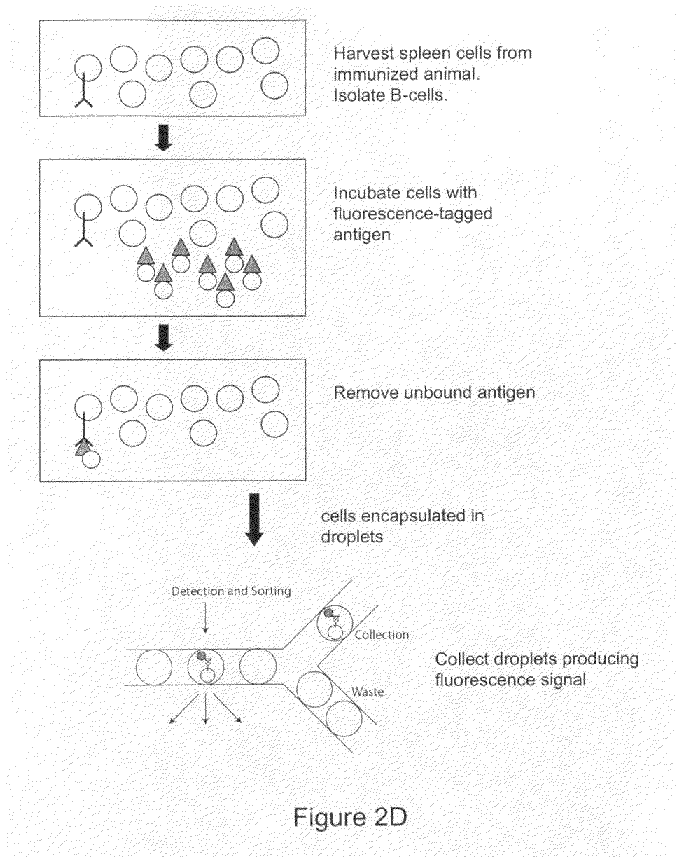

In an exemplary embodiment, the antigen of interest (or an antigenic portion thereof) is attached directly or indirectly to a fluorescent marker, such as fluoroscein isothiocyanate (FITC) or any of a number of fluorescent dye molecules well known in the art, and detected by either a conventional FACS sorter (FIG. 2A) or by an in-line detection system in a microfluidic device (described in detail below).

(B) Microbead-Based Sorting

In one embodiment, cells expressing a desired immunoglobulin are screened and sorted using an antigen immobilized on a solid support, such as microbeads.

Microbeads, also known by those skilled in the art as microspheres, latex particles, beads, or minibeads, are available in diameters from 20 nm to 1 mm and can be made from a variety of materials including silica and a variety of polymers, copolymers and terpolymers including polystyrene (PS), polymethylmethacrylate (PMMA), polyvinyltoluene (PVT), styrene/butadiene (S/B) copolymer, and styrene/vinyltoluene (SNT) copolymer (www.bangslabs.com). They are available with a variety of surface chemistries from hydrophobic surfaces (e.g. plain polystyrene), to very hydrophilic surfaces imparted by a wide variety of functional surface groups: aldehyde, aliphatic amine, amide, aromatic amine, carboxylic acid, chloromethyl, epoxy, hydrazide, hydroxyl, sulfonate and tosyl. The functional groups permit a wide range of covalent coupling reactions for stable or reversible attachment of compounds to the microbead surface.

Microbeads can be directly optically tagged by, for example, incorporating fluorochromes. For example, one hundred different bead sets have been created, each with a unique spectral address due to labeling with precise ratios of red (>650 nm) and orange (585 nm) fluorochromes (Fulton, R. J., McDade, R. L., Smith, P. L., Kienker, L. J. and Kettman, J. R., Jr. (1997), Advanced multiplexed analysis with the FlowMetrix system, Clin Chem, 43, 1749-1756) (www.luminex.com) and sets of up to 10⁶ beads can be encoded by incorporating quantum dots of 10 intensities and 6 colors (Han, M., Gao, X., Su, J. Z., and Nie, S. (2001), Quantum-dot-tagged microbeads for multiplexed optical coding of biomolecules, Nat Biotechnol 19, 631-635 1).

For example, a suitable animal (e.g., rabbit, goat, etc.) may be immunized with a plurality of antigens (e.g., 2 or more antigens). Antibody-producing B cells are then isolated from the spleen, lymph nodes or peripheral blood of the animal. Each antigen (or an immunogenic portion thereof) is attached to a microbead set tagged with a unique detectable marker, so that different antigens are associated with different markers. Antibody-producing B cells can then be simultaneously or sequentially detected and sorted by e.g., FACS, microbeads, or in-line microfluidics. By coding microbead sets with different detectable markers, one can reduce the costs of producing a library of monoclonal antibodies against multiple antigens (e.g., 10 antigens, 100 antigens, 1000 antigens, etc), since fewer animal subjects are needed. Similarly, multiple clones of antibody-producing B cells can be detected and sorted simultaneously or sequentially using antigens tagged with different detectable markers (e.g., fluorescent markers), wherein each antigen (or an antigenic portion thereof) is attached to a different detectable marker. For example, fluorescently-tagged antigens, wherein each antigen is associated with a different fluorescence, can be used to select and sort multiple clones of antibody-producing B cells (e.g., by FACS).

An antigen may be connected to the microbeads either covalently or non-covalently by a variety of means that will be familiar to those skilled in the art (see, for example, (Hermanson, G. T. (1996) Bioconjugate techniques, Academic Press, San Diego)). The antigen may also be attached via a cleavable linker. A variety of such linkers are familiar to those skilled in the art (see, e.g., Gordon, K., and Balasubramanian, S. (1999), Solid phase chemistry—designer linkers for combinatorial chemistry, J Chem Technol Biotechnol 74, 835-851), including for example, linkers which can be cleaved photochemically and reversible covalent bonds which can be controlled by changing the pH (e.g. imines and acylhydrazones), by adjusting the oxido-reductive properties (e.g. disulphides), or using an external catalyst (e.g. cross-metathesis and transamidation).

In certain embodiments, the antigen is immobilized to magnetic beads (e.g., microbeads manufactured by Miltenyi Biotec). FIG. 2B provides an exemplary procedure to screen for B cells expressing a desired immunoglobulin. The antigen (or an immunogenic portion thereof) is attached to magnetic microbeads. The magnetic bead used herein is a particle with magnetism and the diameter is preferably less than 5 microns. Nano-beads (some with a diameter of 50 nm to 230 nm) are also suitable. The magnetic bead is made of suitable magnetic materials and has the advantage of small volume and thus does not affect the reaction hereinafter. Examples of suitable magnetic material include, for example, ferrite, perovskite or chromite.

As shown in FIG. 2B, a plurality of B cells is mixed with the immobilized antigen. The mixing instrument is preferably made of non-magnetic materials, thus that the binding of an antibody to the antigen will not be affected. After mixing for a period of time to allow antibody-antigen binding, a magnetic support is used to provide magnetic field to immobilize the cells that bind to the antigen. Cells without the desired surface immunoglobulin are still homogeneously suspended in the solution. Then, the sample can be washed with an appropriate solution to remove the unbound cells in the presence of the magnetic support. The magnetic support used herein for the immobilization of the magnetic beads includes, for example, a magnetic rack or plate. After removing the unbound cells, the magnetic support may also be removed. In certain embodiments, microbead-bound cells may be detected and sorted by a microfluidic device, as described in detail below.

The immobilized B cells can be directly fused with immortalized cells (e.g., myeloma cells) to generate immortalized immunoglobulin-producing cells (such as hybridomas). Alternatively, the immobilized B cells may be removed from the solid support before immortalization or any other analysis. For example, the antigen may be attached to the solid support via a cleavable linker, so that the immobilized B cells can be cleaved from the solid support.

Optionally, another detectable marker may be used to further select and screen B cells that express a desired immunoglobulin. The detectable marker may be, e.g., a fluorescent marker or a luminescent marker, and may be used before, during, or after the microbead-based sorting.

The immobilized antigen may also be used to initially select cells that bind to the antigen. Afterwards, the antigen-bound B cells are subject to further screening (e.g., by a microfluidic device, as described below, or by FACS).

(C) Microfluidic-Based Sorting

In one aspect, the invention provides a method of screening for cells that expresses a desired immunoglobulin among a repertoire of cells, comprising: a) compartmentalizing the repertoire of cells into microcapsules, such that only a limited number cells (e.g., 1-10 cells) are present in any one microcapsule; and b) detecting the expression of the desired immunoglobulin by the compartmentalized cells; wherein one or both steps a) or b) are performed under microfluidic control. Preferably, only one cell is present in each microcapsule.

(1) Microcapsules

The microcapsules of the present invention preferably have following physical properties. First, cells of each microcapsule are preferably isolated from cells of the surrounding microcapsules, so that there is no or little exchange of cells between the microcapsules over the timescale of the experiment. However, the permeability of the microcapsules may be adjusted such that reagents may be allowed to diffuse into and/or out of the microcapsules if desired. Second, each microcapsule preferably has a limited number of cells per microcapsule. In certain embodiments, each capsule has between one to ten cells. In preferred embodiments, each capsule only has one cell. In the case where the cells are co-encapsulated with a detecting agent, it may also be desirable that there are a limited number of detection molecules per microcapsule. Alternatively, a detecting agent can also be introduced by separate microcapsules, which are then fused to those encapsulating the cells. Third, it is desirable that the formation and the composition of the microcapsules do not significantly affect that viability or antibody-producing activity of the cells.

Consequently, any microencapsulation system used preferably fulfils these properties. The appropriate system(s) may vary depending on the precise nature of the requirements in each application of the invention, as will be apparent to the skilled person.

A wide variety of microencapsulation procedures are available (see e.g., Benita, S. (ed.). (1996) Microencapsulation: methods and industrial applications, Marcel Dekker, New York) and may be used to create the microcapsules used in accordance with the present invention. Indeed, more than 200 microencapsulation methods have been identified in the literature (Finch, C. A. (1993) Encapsulation and controlled release, Spec. Publ.-R. Soc. Chem., vol 138, 35). Additional microencapsulation procedures have been described in U.S. Patent Publication Nos. 2007/0092914 and 2007/0184489, incorporated herein by reference in its entirety.

For example, microcapsules can be generated by interfacial polymerization and interfacial complexation (Whateley, T. L. (1996) Microcapsules: preparation by interfacial polymerisation and interfacial complexation and their applications, in Benita, S. (ed.), Microencapsulation: methods and industrial applications, Marcel Dekker, New York, pp. 349-375). Microcapsules of this sort can have rigid, non-permeable membranes, or semipermeable membranes. Semi-permeable microcapsules bordered by cellulose nitrate membranes, polyamide membranes and lipid-polyamide membranes can all support a variety of biological and biochemical processes (Chang, T. M. (1987) Recycling of NAD(P) by multienzyme systems immobilized by microencapsulation in artificial cells, Methods Enzymol, 136, 67-82; Chang, T. M. S. (1992) Recent advances in artificial cells based on microencapsulation, in Donbrow, M. (ed.), Microcapsules and nanoparticles in medicine and pharmacy, CRC Press, Boca Raton, Fla., pp. 323-339; Lim, F. and Sun, A. M. (1980) Microencapsulated islets as bioartificial endocrine pancreas, Science, 210, 908-910). Alginate/polylysine microcapsules (Lim & Sun, supra), which can be formed under very mild conditions, have also proven to be very biocompatible, providing, for example, an effective method of encapsulating living cells and tissues (Chang, 1992, supra; Sun, A. M., Vasek, I. and Tai, I. (1992) Microencapsulation of living cells and tissues, in Donbrow, M. (ed.), Microencapsulation and nanoparticles in medicine and pharmacy, CRC Press, Boca Raton, Fla., pp. 315-322.).

Non-membranous microencapsulation systems based on phase partitioning of an aqueous environment in a colloidal system, such as an emulsion, may also be used.

Preferably, the microcapsules of the present invention are formed from emulsions; heterogeneous systems of two immiscible liquid phases with one of the phases dispersed in the other as droplets of microscopic or colloidal size (Becher, P. (1957), Emulsions: theory and practice, Reinhold, N.Y.; Sherman, P. (1968) Emulsion science, Academic Press, London; Lissant, K. J. (ed.). (1974) Emulsions and emulsion technology, Marcel Dekker, New York; Lissant, K. J. (ed.). (1984) Emulsions and emulsion technology, Marcel Dekker, New York.).

Emulsions may be produced from any suitable combination of immiscible liquids. Preferably the emulsion of the present invention has water (containing cells and/or biochemical components) as the phase present in the form of finely divided droplets (the disperse, internal or discontinuous phase) and a hydrophobic, immiscible liquid (an “oil”) as the matrix in which these droplets are suspended (the nondisperse, continuous or external phase). Such emulsions are termed “water-in-oil” (W/O). This has the advantage that the entire aqueous phase containing the cells and/or biochemical components is compartmentalized in discreet droplets (the internal phase). The external phase, being a hydrophobic oil, generally does not contain cells or biochemical components and hence is inert.

The emulsion may be stabilized by addition of one or more surface-active agents (surfactants). These surfactants are termed emulsifying agents and act at the water/oil interface to prevent (or at least delay) separation of the phases. Many oils and many emulsifiers can be used for the generation of water-in-oil emulsions; one exemplary compilation listed over 16,000 surfactants, many of which are used as emulsifying agents (Ash, M., Handbook of industrial Surfactants, 1993, Gower Publishing Ltd, Aldershot, England). Suitable oils include light white mineral oil and decane. Suitable surfactants include: non-ionic surfactants (Schick, M. J. (1966), Nonionic surfactants, Marcel Dekker, New York.) such as sorbitan monooleate (Span™80; ICI), sorbitan monostearate (Span™60; ICI), polyoxyethylenesorbitan monooleate (Tween™80; ICI), and octylphenoxyethoxyethanol (Triton X-100); ionic surfactants such as sodium cholate and sodium taurocholate and sodium deoxycholate; chemically inert silicone-based surfactants such as polysiloxane-polycetyl-polyethylene glycol copolymer (Cetyl Dimethicone Copolyol) (e.g. Abil™90; Goldschmidt); and cholesterol.

Emulsions with a fluorocarbon (or perfluorocarbon) continuous phase (Krafft, M. P., Chittofrati, A. and Riess, J. G. (2003) Emulsions and microemulsions with a fluorocarbon phase, Curr. Op. Colloid Interface Sci., 8, 251-258.; Riess, J. G. (2002) Fluorous micro- and nanophases with a biomedical perspective, Tetrahedron, 58, 4113-4131) may be particularly advantageous. For example, stable water-in-perfluorooctyl bromide and water-in-perfluorooctylethane emulsions can be formed using F-alkyl dimorpholinophosphates as surfactants (Sadtler, V. M., Krafft, M. P. and Riess, J. G. (1996) Achieving stable, reverse water-in fluorocarbon emulsions, Angew. Chem. Int. Ed. Engl., 35, 1976-1978). Non-fluorinated compounds are essentially insoluble in fluorocarbons and perfluorocarbons (Curran, D. P. (1998) Strategy-level separations in organic synthesis: from planning to practice, Angew Chem Int Ed, 37, 1174-1196; Hildebrand, J. H. and Cochran, D. F. R. (1949) J. Am. Chem. Soc., 71, 22; Hudlicky, M. (1992) Chemistry of Organic Fluorine Compounds, Ellis Horwood, N.Y.; Scott, R. L. (1948) J. Am. Chem. Soc., 70, 4090; Studer, A., Hadida, S., Ferritto, R., Kim, S. Y., Jeger, P., Wipf, P. and Curran, D. P. (1997) Fluorous synthesis: a fluorous-phase strategy for improving separation efficiency in organic synthesis, Science, 275, 823-826) and small drug-like molecules (typically <500 Da and Log P<5) (Lipinski, C. A., Lombardo, F., Dominy, B. W. and Feeney, P. J. (2001) Experimental and computational approaches to estimate solubility and permeability in drug discovery and development settings, Adv Drug Deliv Rev, 46, 3-26) are compartmentalized very effectively in the aqueous microcapsules of water-in-fluorocarbon and water-in-perfluorocarbon emulsions—with little or no exchange between microcapsules.

Creation of an emulsion generally requires the application of mechanical energy to force the phases together. There are a variety of ways of doing this which utilize a variety of mechanical devices, including stirrers (such as magnetic stir-bars, propeller and turbine stirrers, paddle devices and whisks), homogenizers (including rotor-stator homogenizers, high-pressure valve homogenizers and jet homogenizers), colloid mills, ultrasound and “membrane emulsification” devices (Becher, 1957, supra; Dickinson, E. (1994) Emulsions and droplet size control, in Wedlock, D. J. (ed.), Controlled particle, droplet and bubble formation), and microfluidic devices (Umbanhowar, P. B., Prasad, V. and Weitz, D. A. (2000) Monodisperse emulsions generated via drop break off in a coflowing steam, Langmuir, 16, 347-351).

The technology exists to create emulsions with volumes all the way up to industrial scales of thousands of liters (Becher, 1957, supra; Sherman, 1968, supra; Lissant, 1974, supra; Lissant, 1984, supra).

The preferred microcapsule size will vary depending upon the precise requirements of any individual screening process that is to be performed according to the invention. In all cases, there will be an optimal balance between the size of the repertoire of the cells and the sensitivities of the assays to determine the presence of a desired immunoglobulin.

The size of emulsion microcapsules may be varied simply by tailoring the emulsion conditions used to form the emulsion according to requirements of the screening system. The larger the microcapsule size, the larger is the volume that will be required to encapsulate a given repertoire of the cells, since the ultimately limiting factor will be the size of the microcapsule and thus the number of microcapsules possible per unit volume.

Water-in-oil emulsions can be re-emulsified to create water-in-oil-in water double emulsions with an external (continuous) aqueous phase. These double emulsions can be analyzed and, optionally, sorted by fluorescence, luminescence, or other detectable markers (Bernath, K., Hai, M., Mastrobattista, E., Griffiths, A. D., Magdassi, S, and Tawfik, D. S. (2004) In vitro compartmentalization by double emulsions: sorting and gene enrichment by fluorescence activated cell sorting, Anal Biochem, 325, 151-157).

Highly monodisperse microcapsules can be produced using microfluidic techniques. For example, water-in-oil emulsions with less than 3% polydispersity can be generated by droplet break off in a co-flowing steam of oil (Umbanhowar et al., Monodisperse emulsions generated via drop break off in a coflowing steam, Langmuir, 16, 347-351, 2000). Microfluidic systems can also be used for laminar-flow of aqueous microdroplets dispersed in a stream of oil in microfluidic channels (Thorsen, T., R. W., R., Arnold, F. H. and Quake, S. R. (2001) Dynamic pattern formation in a vesicle-generating microfluidic device, Phys. Rev. Letts., 86, 4163-4166). This allows the construction of microfluidic devices for flow analysis and, optionally, flow sorting of microdroplets (Fu, A. Y., Chou, H. P., Spence, C., Arnold, F. H. and Quake, S. R. (2002) An integrated microfabricated cell sorter, Anal Chem, 74, 2451-2457).

Advantageously, highly monodisperse microcapsules can be formed using systems and methods for the electronic control of fluidic species. One aspect of the invention relates to systems and methods for producing droplets of fluid surrounded by a liquid. The fluid and the liquid may be essentially immiscible in many cases, i.e., immiscible on a time scale of interest (e.g., the time it takes a fluidic droplet to be transported through a particular system or device). In certain cases, the droplets may each be substantially the same shape or size, as further described below. The fluid may also contain other species, for example, certain molecular species (e.g., as further discussed below), cells, particles, etc.

In certain embodiments, electric charge may be created on a fluid surrounded by a liquid, which may cause the fluid to separate into individual droplets within the liquid. In certain embodiments, the fluid and the liquid may be present in a channel, e.g., a microfluidic channel, or other constricted space that facilitates application of an electric field to the fluid (which may be “AC” or alternating current, “DC” or direct current etc.), for example, by limiting movement of the fluid with respect to the liquid. Thus, the fluid can be present as a series of individual charged and/or electrically inducible droplets within the liquid. In one embodiment, the electric force exerted on the fluidic droplet may be large enough to cause the droplet to move within the liquid. In some cases, the electric force exerted on the fluidic droplet may be used to direct a desired motion of the droplet within the liquid, for example, to or within a channel or a microfluidic channel. Electric charge may be created in the fluid within the liquid using any suitable technique, for example, by placing the fluid within an electric field (which may be AC, DC, etc.), and/or causing a reaction to occur that causes the fluid to have an electric charge, for example, a chemical reaction, an ionic reaction, a photocatalyzed reaction, etc., all of which are known in the art.

In some embodiments, the fluidic droplets may each be substantially the same shape and/or size. The shape and/or size can be determined, for example, by measuring the average diameter or other characteristic dimension of the droplets.

Microcapsules can further be fused or split. For example, aqueous microdroplets can be merged and split using microfluidics systems (Link, D. R., Anna, S. L., Weitz, D. A. and Stone, H. A. (2004) Geometrically mediated breakup of drops in microfluidic devices, Phys. Rev. Letts., 92, 054503; Song, H. and Ismagilov, R. F. (2003) Millisecond kinetics on a microfluidic chip using nanoliters of reagents, J Am Chem Soc, 125, 14613-14619; Song, H., Tice, J. D. and Ismagilov, R. F. (2003) A microfluidic system for controlling reaction networks in time, Angew. Chem. Int. Ed. Engl., 42, 767-772.). Microcapsule fusion allows the mixing of reagents. Fusion, for example, of a first microcapsule containing a cell with a second microcapsule containing a detecting agent to detect the expression of a desired immunoglobulin could facilitate high throughput screening and sorting. Microcapsule splitting allows single microcapsules to be split into two or more smaller microcapsules. For example a single microcapsule containing several cells (e.g., B cells) can be split into multiple microcapsules, each having a single cell, which can then each be fused with a different microcapsule containing another type of cell (e.g., a myeloma cell) to create a hybridoma fusion.

(2) Screening and Sorting of Microcapsules

In still another aspect, the invention provides systems and methods for screening or sorting fluidic droplets in a liquid, and in some cases, at relatively high rates. For example, a droplet encapsulating one or more cells expressing a desired immunoglobulin may be sensed and/or determined. Detecting these cell surface immunoglobulins can be done in any number of ways, such as enzymatic, magnetic, luminescence or fluorescence analysis. After detection, the droplet may be directed towards a particular region of the device, for example, for, sorting or collecting purposes.

In certain embodiments, a single cell suspension of the immunized spleen, lymph node, blood or other tissue is first generated. The antigen (optionally linked to a detectable marker) is then added for the desired time to allow antibody-antigen binding, followed by washing (e.g., by several rounds of centrifugation of the cells) to remove unbound antigen molecules. The cells can then be encapsulated. The presence of the antigen on the surface of certain B cells can be detected, for example, by in-line sensors of fluorescence, light, or absorption.

In certain embodiments, droplets having one or more cells expressing a desired immunoglobulin are screening by a detecting agent. In certain embodiments, the detecting agent is an antigen or immunogenic portion thereof that is specifically recognized by the desired immunoglobulin. In certain embodiments, the antigen is attached to a detectable marker. In certain embodiments, the detectable marker is a fluorescent marker, a luminescent marker, a chromogenic marker, or a magnetic marker.

In certain embodiments, the detectable marker is covalently linked to or fused with the antigen. For example, the antigen may be fused to an enzyme, such as beta-galactosidase, alkaline phosphatase, or horseradish peroxidase. There are several advantages of using such enzyme-based detection methods. First, encapsulation allows the reactions to run for the desired time. Furthermore, the reaction products accumulate in the confines of the microcapsules. Thus, such detection methods can be highly sensitive—at the level of tens of molecules per cell. In addition, many enzymes have a host of substrates that can generate fluorescent or luminescent products for detection.

Alternatively, an antigen covalently linked to or fused with a fluorescent protein (e.g., green fluorescence protein (GFP)) can be used to facilitate the detection of antigen-bound B cells.

In certain embodiments, after incubation with the antigen to allow antigen binding, the cells are mixed with a detecting agent, and the detecting agent and the cells are coencapsulated. Alternatively, a detecting agent can also be introduced by separate microcapsules, which are fused with those microcapsules encapsulating the cells.

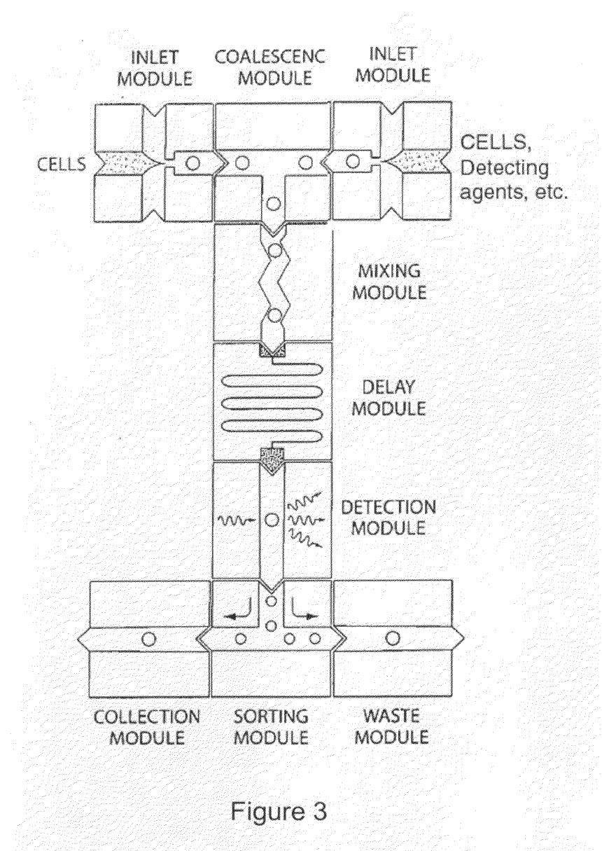

FIG. 2 provides exemplary procedures for detecting and selecting B cells that express a desired immunoglobulin. A repertoire of B cells, either harvested from spleen cells from an immunized animal, or isolated from a biological sample (e.g., blood) from a subject who has been exposed to an antigen, is pre-mixed with the antigen before the microcapsules are made. In FIG. 2C, the antigen, or an immunogenic portion thereof, is attached to an enzyme, (e.g., beta-galactosidase, alkaline phosphatase, or horseradish peroxidase). After incubation to allow antibody-antigen binding, excess, unbound antigen molecules are removed. In one option, enzyme substrates that can generate fluorescent or luminescent products are mixed with the B cells prior to encapsulation. Microcapsules, each containing a single B cell (and an enzyme substrate), are then formed using a microfluidic device. In another option, microcapsules, each containing a single B cell are first formed; an enzyme substrate is then introduced as separate microcapsules, which are subsequently fused to microcapsules encapsulating B cells. The reactions are allowed to run for the desired time (which can be done, for example, in a mixing module and/or a delay module of a microfluidic device, see e.g., FIG. 3). Afterwards, the microcapsules are passed through a detection module of a microfluidic device. Microcapsules having antigen-bound B cells are detected by the detectable marker (e.g., luminescence or fluorescence) and selected for collection, and microcapsules having non-antigen-bound B cells are directed to “waste.” In FIG. 2D, the antigen, or its immunogenic portion thereof, is attached to a fluorescence marker. After incubation to allow antibody-antigen binding, excess, unbound antigen molecules are removed. Microcapsules, each containing a single B cell, are then formed using a microfluidic device. Antigen-bound B cells are detected by the fluorescence and selected for collection.

In certain embodiments, the microcapsules can be sorted using either a microfluidic or conventional fluorescence-activated cell sorter (Norman, Med. Phys., 1980 November-December; 7(6):609-15.; Mackenzie and Pinder, Dev. Biol. Stand. 1986, 64:181-93), or similar device. For example, fluorescence-activated cell sorting equipment from established manufacturers (e.g. Becton-Dickinson, Coulter, Cytomation) allows the analysis and sorting at up to 100,000 microcapsules per second. In addition, the fluorescence signal from each microcapsule corresponds tightly to the number of fluorescent molecules present. As little as few hundred fluorescent molecules per microcapsules can be quantitatively detected. Also, the wide dynamic range of the fluorescence detectors (typically 4 log units) allows easy setting of the stringency of the sorting procedure, thus allowing the recovery of the optimal number microcapsules from the starting pool (the gates can be set to separate microcapsules with small differences in fluorescence or to only separate out microcapsules with large differences in fluorescence, dependant on the selection being performed). Finally, fluorescence-activated cell sorting equipment can perform simultaneous excitation and detection at multiple wavelengths (Shapiro, H. M. (1995), Practical Flow Cytometry, 3 ed. (New York, Wiley-Liss) allowing positive and negative selections to be performed simultaneously by monitoring the labeling of the microcapsules with two to thirteen (or more) fluorescent markers, for example, if substrates for two alternative targets are labeled with different fluorescent tags the microcapsules can labeled with different fluorophores dependent on the target regulated.

The method of the invention permits the rapid identification of even a very small number of cells that expresses a desired immunoglobulin in a repertoire of cells. Since the expression of membrane-bound immunoglobulins (in particular IgGs) is relatively low, the methods allow quick and sensitive detection of desired B cells.

(3) Microfluidic Systems

In certain embodiments, microcapsules of the invention can be formed, screened, and/or sorted using microfluidic devices. A microfluidic device typically refers to a system or device having fluidic conduits, such as channels, that are typically fabricated at the micron to submicron scale. Generally, the fluidic conduits have at least one cross-sectional dimension of less than 1 mm (preferably in the range of from about 0.1 μm to about 500 μm), and a ration of length to largest cross-sectional dimension of at least 3:1. The term “microfluidic control” refers to the use of a microfluidic system comprising microfluidic channels as defined herein to direct or otherwise control the formation and/or movement of microcapsules (or “droplets”) in order to carry out the methods of the present invention. For example, “microfluidic control” of microcapsule formation refers to the creation of microcapsules using a microfluidic device to form “droplets” of fluid within a second fluid, thus creating a microcapsule. Microcapsules sorted under microfluidic control are sorted, as described herein, using a microfluidic device to perform one or more of the functions associated with the sorting procedure.

Although exemplary microfluidic devices suitable for the invention are described briefly below, methods of employing microfluidics for cell delivery are well-known in the art and are described, for example, in Love, et al., MRS Bulletin, pages 523-27 (2001); Delamarche et al,; Journal of American Chemical Society, Vol. 120, pages 500-08 (1998), Delamarche et al,; Science, Vol. 276, pages 779-81 (1997), Quake et al., Science, Vol. 290, pages 1536-40 (2000), and International Publication Nos. WO07/081,385, WO 07/081,386, WO 07/133,710, each hereby incorporated by reference in its entirety.

One type of microfluidic devices described herein is based on the creation and electrical manipulation of aqueous phase microcapsules (droplets) completely encapsulated by an inert fluorocarbon oil stream. This combination enables electrically addressable droplet generation, highly efficient droplet coalescence, precision droplet breaking and recharging, and controllable single droplet sorting. Additional passive modules include multi-stream droplet formulations, mixing modules, and precision break-up modules. The integration of these modules provides an enabling technology for a droplet based, high-throughput microfluidic reactor system.

The microfluidic devices can use a flow-focusing geometry to form the droplets. For example, a water stream can be infused from one channel through a narrow constriction; counter propagating oil streams (preferably fluorinated oil) hydrodynamically focus the water stream and stabilize its breakup into micron size droplets as it passes through the constriction. In order to form droplets, the viscous forces applied by the oil to the water stream should overcome the water surface tension. The generation rate, spacing and size of the water droplets is controlled by the relative flow rates of the oil and the water streams and nozzle geometry. While this emulsification technology is extremely robust, droplet size and rate are tightly coupled to the fluid flow rates and channel dimensions. Moreover, the timing and phase of the droplet production cannot be controlled. In certain embodiments, the microfluidic devices of the present invention overcome these limitations by incorporating integrated electric fields, thereby creating an electrically addressable emulsification system. In one embodiment, this can be achieved by applying high voltage to the aqueous stream and charge the oil water interface. The water stream behaves as a conductor while the oil is an insulator; electrochemical reactions charge the fluid interface like a capacitor. At snap-off, charge on the interface remains on the droplet. The droplet size decreases with increasing field strength. At low applied voltages the electric field has a negligible effect, and droplet formation is driven exclusively by the competition between surface tension and viscous flow.

The microfluidic, droplet-based reaction-confinement system can further include a mixer which combines two or more reagents to initiate a biological or chemical reaction. Multi-component droplets can easily be generated by bringing together streams of materials at the point where droplets are made.

In droplet-based microfluidic devices, multi-component droplets may also be accomplished by combining (or coalescing) different droplets, each containing an individual component. For example, placing electrostatic charge of opposite sign on each droplet, and applying an electric field can force droplets to coalesce. The microfluidic device may include two separate nozzles that generate droplets with different compositions and opposite charges. The droplets are brought together at the confluence of the two streams. The electrodes used to charge the droplets upon formation also provide the electric field to force the droplets across the stream lines, leading to coalescence. In the absence of an electric field, droplets in the two streams may not arrive at the point of confluence at exactly the same time. When they do arrive synchronously the oil layer separating the droplets may not drain quickly enough to facilitate coalescence and as a result the droplets do not coalesce. Thus, upon application of an electric field, droplet formation becomes exactly synchronized, ensuring that droplets each reach the point of confluence simultaneously (i.e., paired droplets).

Moreover, since the droplets are oppositely charged they are attracted to one another, which forces them to traverse the fluid stream lines and contact each other, thereby causing them to coalesce. The remarkable synchronization of the droplet formation results from coupling of the break-off of each of the pair of droplets as mediated by the electric field. The use of oppositely charged droplets and an electric field to combine and mix reagents is extremely robust, and 100% of the droplets coalesce with their partner from the opposite stream.

Other embodiments of the microfluidic devices can include a droplet sorter, in which the contents of individual droplets are probed, and selected droplets sorted into discrete streams. In one embodiment, such sorting in microfluidic devices can be accomplished through the use of mechanical valves. In another embodiment, the use of electrostatic charging of droplets provides an alternate means that can be precisely controlled, can be switched at high frequencies, and requires no moving parts. Electrostatic charge on the droplets enables drop-by-drop sorting based on the linear coupling of charge to an external electric field. As an example, a T-junction bifurcation that splits the flow of carrier fluid equally will also randomly split the droplet population equally into the two streams. However, a small electric field applied at the bifurcation precisely dictates which channel the drops enter. Varying the direction of the field varies the direction of the sorted droplets. The large forces that can be imparted on the droplets and the short time required to switch the field make this a fast and robust sorting engine with no moving parts; thus the processing rate is limited only by the rate of droplet generation and electric field switching time, and can easily exceed 20,000 per second.

FIG. 3 illustrates an example of a microfluidic delivery device to deliver a repertoire of cells. The microfluidic device of FIG. 3 comprises: (a) a microfabricated substrate comprising at least one inlet channel adapted to carry at least one dispersed phase fluid and at least one main channel adapted to carry a continuous phase fluid, where inlet channel is in fluid communication with the main channel at one or more inlet modules such that the dispersed phase fluid is immiscible with the continuous phase fluid and forms a plurality of droplets in the continuous phase fluid; (b) an optional coalescence module, where an electric field is applied to cause two or more droplets to coalesce; and (c) a detection module including a detection apparatus for evaluating the contents and/or characteristics of the coalesced droplets produced in the coalescence module. The microfabricated substrate can further comprise one or more sorting modules, collection modules, waste modules, branch channels, delay modules, mixing modules and/or UV release modules, or any combinations thereof in any order.

In certain embodiments, a droplet encapsulating one or more cells expressing a desired immunoglobulin may be sensed and/or determined, for example, by luminescence or fluorescence, and, in response, an electric field may be applied or removed from the fluidic droplet to direct the fluidic droplet to a particular region (e.g. a channel).

In certain embodiments, a fluidic droplet may be directed by creating an electric charge (e.g., as previously described) on the droplet, and steering the droplet using an applied electric field, which may be an AC field, a DC field, etc.

In certain embodiments, a fluidic droplet may be sorted or steered by inducing a dipole in the fluidic droplet (which may be initially charged or uncharged), and sorting or steering the droplet using an applied electric field.

In certain embodiments, the fluidic droplets may be screened or sorted within a fluidic system by altering the flow of the liquid containing the droplets. For instance, a fluidic droplet may be steered or sorted by directing the liquid surrounding the fluidic droplet into a first channel, a second channel, etc.

In certain embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled.

The methods for producing microfluidic device are known in the art. The method of producing a microfluidic device generally comprises one or more of the following steps in any combination: 1) hard lithography, 2) soft lithography, 3) extraction and/or punch though, 4) bonding, 5) channel coating, 6) interconnect assembly, 7) electrode injection and 8) waveguide injection and fiber installation. The steps have been described in detail, for example, in International Publication Nos. WO07/081,385, WO 07/081,386 and WO 07/133,710.

Creating and manipulating microcapsules in microfluidic systems have several advantages. First, stable streams of microcapsules can be formed in microchannels and identified by their relative positions. Second, if the reactions are accompanied by an optical signal (e.g. a change in luminescence or fluorescence), a spatially-resolved optical image of the microfluidic network allows time resolved measurements of the reactions in each microcapsule. And finally, microcapsules can be separated using a microfluidic flow sorter to allow recovery and further analysis or manipulation of the molecules they contain.

In certain embodiments, the microfluidic device may be used to screen for cells that expresses a desired immunoglobulin among a repertoire of cells. The method includes: a) providing a microfabricated substrate comprising at least one inlet channel adapted to carry at least one dispersed phase fluid and at least one main channel adapted to carry a continuous phase fluid, where the inlet channel is in fluid communication with the main channel at one or more inlet modules, and where the dispersed phase fluid is immiscible with the continuous phase fluid; b) flowing a first dispersed phase fluid comprising the repertoire of cells through a first inlet channel such that the first dispersed phase fluid resides as one or more droplets in the continuous phase fluid; c) flowing at least a second dispersed phase fluid comprising a detecting agent (such a detectable marker that specifically recognizes antigen-bound B cells) through a second inlet channel such that the second dispersed phase fluid resides as one or more droplets in the continuous phase fluid; d) slowing or stopping at least one droplet formed in step (b) by exerting a dielectrophoretic force onto the droplet; e) coalescing at least one droplet formed in step (c) with the droplet slowed or stopped in step (d) under the influence of an electric field within a coalescence module, thereby producing a nanoreactor; f) incubating the nanoreactor within a delay module; and g) interrogating the nanoreactor for a predetermined characteristic of the cells (such as fluorescence) within a detection module. For example, an antigen attached to an enzyme will only bind to those B cells expressing an immunoglobulin that recognizes the antigen. After incubation to allow antibody-antigen binding, unbound antigen molecules may be subsequently removed, and cells may be passed through the first inlet channel to form microcapsules. A fluorescent or luminescent substrate of the enzyme can be passed through the second inlet channel to form microcapsules. A microcapsule containing a cell and a microcapsule containing the detecting agent then coalesce in the coalesce module. After incubation in the delay module to allow fluorescence or luminescence to develop, the microcapsules are passed through a detection module to select B cells that express a desired immunoglobulin. See, e.g., FIG. 3.

### 4. Immortalizing Antibody-Producing Cells

Once cells expressing a desired immunoglobulin have been identified, the cells can then be immortalized by any of a number of standard means. For example, hybridoma cells may be prepared by fusing the identified cells with an immortalized cell line according to the well-known well-established hybridoma technology. See e.g., Nature 265: 495-97 (1975); Kohler and Milstein, Eur. J. Immunol. 6: 511 (1976); see also, CURRENT PROTOCOLS IN MOLECULAR BIOLOGY, Ausubel et al. Eds. (1989).

Preferred immortalized cell lines are those that fuse efficiently, support stable high level expression of antibody by the selected antibody-producing cells, and are sensitive to a medium such as HAT medium. More preferred immortalized cell lines are hypoxanthine guanine phosphoribosyl transferase (HGPRT) deficient cell lines, such as myeloma cells, HGPRT-293T cells, etc. Murine myeloma lines can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. See, e.g., Kozbor, J. Immunol., 133:3001 (1984); Brodeur et al., Monoclonal Antibody Production Techniques and Applications, Marcel Dekker, Inc., New York, (1987) pp. 51-63.

Other methods of immortalizing cells include, but are not limited to, transfecting them with oncogenes, infecting them with an oncogenic virus and cultivating them under conditions that select for immortalized cells, subjecting them to carcinogenic or mutating compounds, and inactivating a tumor suppressor gene. See, e.g., Harlow and Lane, supra.

If fusion with myeloma cells is used, the myeloma cells preferably do not secrete immunoglobulin polypeptides (a non-secretory cell line). Typically the antibody producing cell and the immortalized cell (such as but not limited to myeloma cells) with which it is fused are from the same species. Rabbit fusion hybridomas, for example, may be produced as described in U.S. Pat. No. 5,675,063, C. Knight, issued Oct. 7, 1997. The immortalized antibody producing cells, such as hybridoma cells, are then grown in a suitable selection media, such as HAT, and the supernatant can be screened for monoclonal antibodies having the desired specificity. The secreted antibody may be recovered from tissue culture supernatant by conventional methods such as precipitation, ion exchange or affinity chromatography, or the like.

Traditionally, hybridoma cells are produced by fusing a population of spleen cells with myeloma cells; afterwards, the hybridoma cells are screened for antibody production and antibody specificity. Because only a small percentage of the spleen cells express the desired immunoglobulin, the typical fusion involves 100 million spleen cells, which yields about 1000 hybridomas, among which five to 10 are positive for a particular antigen. The invention provides novel methods that significantly improve the efficiency of hybridoma screening procedure, as B cells expressing a desired immunoglobulin are selected prior to immortalization (such as fusion with myeloma), thereby improving fusion efficiency and reducing the labor and costs for hybridoma screening.

In certain embodiment, the overall fusion efficiency is determined first. After screening (e.g., by FACS, by microbeads, or by microfluidics, as described herein), each B cell expressing a desired immunoglobulin as a cell-surface bound immunoglobulin is placed a microtiter well, together with the requisite number of immortalized cells (e.g., myeloma cells), as determined by the fusion efficiency, to produce an immortalized immunoglobulin-producing cell (e.g., a hybridoma cell). For example, if the overall fusion efficiency is approximately 1 B cell:20 myeloma cells, each B cell expressing a desired immunoglobulin will be placed a microtiter well, together with 20 myeloma cells, to produce a hybridoma cell.

In certain embodiments, microfluidic devices, such as those described above, are used to create immortalized antibody-producing cells.

In certain embodiments, a microfluidic device is used to produce an immortalized immunoglobulin-producing cell (e.g., a hybridoma cell). The method comprises: a) providing a microfabricated substrate comprising at least one inlet channel adapted to carry at least one dispersed phase fluid and at least one main channel adapted to carry a continuous phase fluid, where the inlet channel is in fluid communication with the main channel at one or more inlet modules, and where the dispersed phase fluid is immiscible with the continuous phase fluid; b) flowing a first dispersed phase fluid comprising a plurality of B cells expressing a desired immunoglobulin, through a first inlet channel such that the first dispersed phase fluid resides as one or more droplets in the continuous phase fluid; c) flowing at least a second dispersed phase fluid comprising a plurality of immortalized cells (e.g., myeloma cells) through a second inlet channel such that the second dispersed phase fluid resides as one or more droplets in the continuous phase fluid; d) slowing or stopping at least one droplet formed in step (b) by exerting a dielectrophoretic force onto the droplet; e) coalescing at least one droplet formed in step (c) with the droplet slowed or stopped in step (d) under the influence of an electric field within a coalescence module, thereby producing a nanoreactor; f) incubating the nanoreactor within a delay module to cause the fusion of a B cell with an immortalized cell (e.g., a myeloma cell); and g) interrogating the nanoreactor for fusion cells. See, FIG. 3. Slowing or stopping the droplets from step (b) allows pairing of the droplets from step (c) before they move to the location (e) where they are driven to coalesce by an electric field, or passively by passing through a narrowing of the channel. In step (d), the pairing of droplets from (b) and (c) may be achieved in one of three ways: (i) using the dielectrophoretic force produced by the electric field gradient; (ii) using droplets of two different sizes, which works best when one droplet is comparable to the channel width and one droplet is smaller than the channel width, so that the smaller droplet catches up to the larger droplet; and (iii) the droplet in steps (b) and (c) have different viscosities, and thus, move at different velocities. The droplets may be of different sizes; for example, the larger droplet has enough volume so that it would have a diameter greater than the channel width if it were spherical.

The term “nanoreactor” encompasses “droplet”, “nanodrop”, “nanodroplet”, “microdrop,” “microdroplet”, or “microcapsules,” as well as an integrated system for the manipulation and probing of droplets, as described in detail herein. Nanoreactors as described herein can be 0.1-1000 μm (e.g., 0.1, 0.2 . . . 5, 10, 15, 20, 25, 30, 35, 40, 45, 50 . . . 1000) in diameter, or any size within in this range. Droplets at these dimensions tend to conform to the size and shape of the channels, while maintaining their respective volumes. Thus, as droplets move from a wider channel to a narrower channel they become longer and thinner, and vice versa.

In certain embodiments, each droplet formed in the first inlet channel contains a single B cell, and each droplet formed in the second inlet channel contains a single immortalized cell (e.g., a myeloma cell). The traditional fusion techniques often generate large numbers of inappropriate fusion products (such as myeloma-myeloma fusions, B cell-B cell fusions; multiple myeloma-single B cell fusions; or single myeloma-multiple B cell fusions) that are non-viable. This embodiment of the invention, which places a single B cell in close contact with a single immortalized cell, avoid the inefficiency of the traditional fusion methods. In addition, because this embodiment of the invention places a single B cell in close contact with a single immortalized cell, the use of HGPRT-deficient cells become unnecessary. Traditionally, the use of HAT medium and HGPRT-deficient cells ensures that only the B cell-myeloma hybrids survive (inappropriate myeloma-myeloma fusion products will die, as they cannot produce nucleotides by de novo or salvage pathway; inappropriate B cell-B cell fusion products will also die as they have a short life span). In contrast, by placing a single B cell in close contact with a single immortalized cell, inappropriate fusion products will not be formed. Therefore, any immortalized cells with a selection marker can be used as a fusion partner.

A number of methods may be used for carrying out cell-cell fusion in vitro, including the use of chemicals such as polyethylene glycol (PEG), the use of focused laser beams (laser-induced fusion), the application of pulsed electric fields (electrofusion), and the use of fusogenic proteins.

In certain embodiments, a microfluidic device is used for flow-through electrofusion of cells based on applied voltage (e.g., constant direct current). The fluidic device uses electric field to provide for high throughput cell electropermeabilization, cell lysis, or cell electrofusion. The cells may be electrofused while flowing through the geometrically defined narrow electrofusion section. Preferably, cells are electrofused with a high survival rate. Thus, overall voltage may be controlled so that only the field in the narrow section(s) is high enough for cell fusion and the field in the rest of the channel is too weak to have adverse effects on the cell viability. When cells flow through the device, they experience field intensity variations equivalent to electrical pulse(s).

Because the microfluidic device produces nanoreactors, fusion partners are brought into contact with each other. This means either that they are placed so that the outer surfaces of the fusion partners are touching, or that the fusion partners are placed at a very small distance from each other. The fact that the two fusion partners are placed next to each other before fusion makes it possible to avoid dielectrophoresis, which traditionally is used for the creation of close contact between cells.

The electrical field may be obtained by use of a low-voltage or high-voltage pulse-generator depending on the electrodes used, and other experimental parameters. The voltage generator is used to produce an electric field strength sufficient to result in fusion between the two fusion partners, as well as pore formation in one cell structure.

The electrical filed may be provided by one or two microelectrodes positioned close to the two fusion partners from the cellular membrane. Microelectrodes may be electrodes of cellular to subcellular dimensions.

The electrodes can be made of a solid electrically conducting material, or they can be hollow for delivery of different chemical agents into the fusion container. The electrodes can be made from different materials. A special type of electrodes are hollow and made from fused silica capillaries of a type that frequently is used for capillary electrophoresis and gas chromatographic separations. These capillaries are typically one to one hundred micrometers in inner diameter, and five-to-four hundred micrometers in outer diameter, with lengths between a few millimeters up to one meter. For cell fusion applications, these electrodes are filled with an electrolyte, preferably a physiological buffer solution.

The electrodes may be a movable type that can be positioned at will close to a cell or a cell-like structure. Preferably such electrodes are controlled by micromanipulators. The electrodes can also be fabricated directly on chip. Such electrodes can be movable in a microelectromechanical device but they can also be stationary. Electrodes can be fabricated on-chip in a variety of materials. For example, metal electrodes can be deposited on silicon using evaporation or sputtering. Furthermore, it is preferred to provide the fusion partners in an electrofusion buffer in the sample containers.

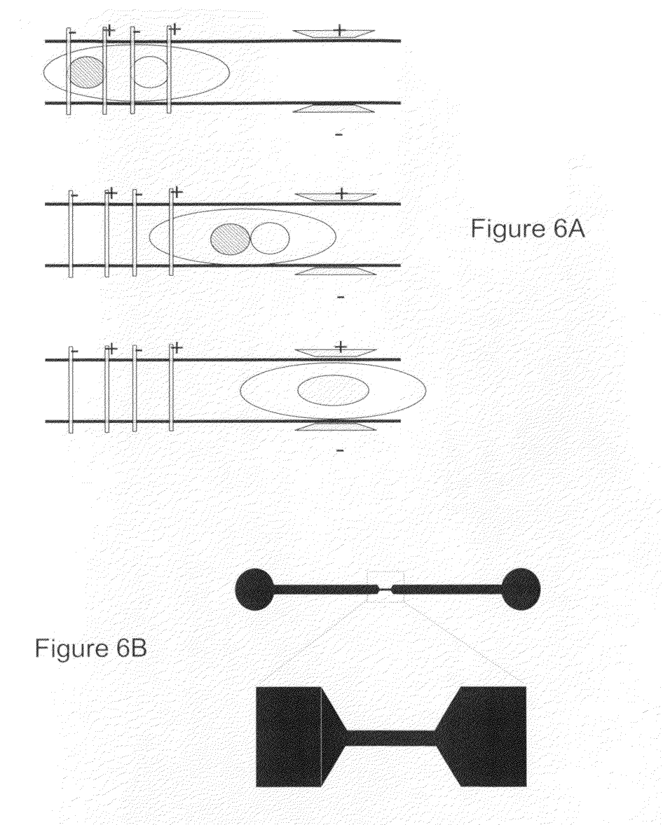

FIGS. 6A and 6B illustrate a few different means of electrofusion by which non-immortalized B cells can be fused with other cells, such as myeloma cells and other cells to create an immortalized cell line producing the antibody that the B cell was selected for. Electrofusion is based on the application of high-voltage electric pulses, and can be applied to a wide range of cell types with high efficiency and high post-fusion viability.

In one representative example, shown in FIG. 6A, non-immortalized B cells and myeloma cells are encapsulated together in a droplet. As the droplet passes through an electrofusion chamber, a first set of electrodes generates a dipole moment in each cell such that the cells are attracted to one another. The cell-containing droplet then encounters a second set of electrodes that induce electrofusion, preferably by a high-voltage direct current (DC) pulse, of the two cells.

In another one example, adapted from Wang et al. 2006 Applied Physics Letters 89:234102, the cells are first conjugated based on biotin-streptavidin interaction. The electrofusion is then conducted by flowing the linked cells through a microfluidic channel with geometric variation under continuous DC voltage. As shown in FIG. 6B, an electrofusion device of this design cam include a microfluidic channel with narrow and wide sections. Devices with one or multiple narrow sections can be used to induce fusion of the cells. In certain embodiments, the field strength at the center of the narrow section is around 9-10 times higher than the field strength in the bulk of the wide sections. This number is roughly the ratio between the width in the wide section and the one in the narrow section. The overall voltage can be controlled so that only the field in the narrow section is high enough for cell fusion and the field in the rest of the channel is too weak to have adverse effects on the viability of cells. When cells flow through the device, they experience field intensity variations equivalent to electrical pulse. The “pulse width” is determined by the length of the narrow section and the velocity of cells. In certain embodiments, these fusion chambers can be generated as microfluidic chips, fabricated for example based on polydimethylsiloxane using standard soft lithography method.

Still another device that can be adapted for use in the present invention is described in Skelley et al. (2009) Nature Methods, 6(2): 147-152. Briefly, this type of device traps cells in micrometer-sized capture cups (2 cells at a time) on a chip and efficiently achieves cell pairing and subsequent chemically or electrically induced fusion. To illustrate, first the B cells can be flowed across the chip in one direction and caught in traps that are large enough to hold only one cell. Once the cells are trapped, liquid is flowed across the chip in the opposite direction, pushing the cells out of the small cups and into larger cups across from the small ones. Once one B cell is in each large cup, myeloma cells can flowed into the large cups. Each cup can only hold two cells, so each ends up with one B cell and myeloma cell. After the cells are paired in the traps, they can be joined together by an electric pulse that fuses the cell membranes.

In addition to electrofusion, the microfluidic devices may be used as a platform for chemical-induced fusion (e.g., PEG), or integrating chemical-induced fusion and electrofusion. The presence of a small amount of PEG, for example, can increase the success of electrofusion. It is also well known that a number of chemical and biological factors affect the yield of fusion. The osmolality of the medium is one example; fusion yields, for both electrofusion and PEG-induced fusion, increases greatly if carried out in hypotonic media or if cells were treated briefly in hypotonic media then returned to isotonic media for fusion. With microfluidics, varying the osmolality or the concentration of the fusion media inside the channels can be conveniently performed. The presence of divalent cations (e.g. calcium) and pretreatment of cells with protease are other examples in which fusion yield can be improved dramatically. These chemical and biological conditions, however, vary widely between cells types and the best conditions for carrying out fusion need to be found empirically. In the bulk fusion format, varying these parameters is tedious, whereas microfluidics provides a natural platform for rapidly testing the suitability of each of these conditions. Furthermore, the electrofused or electroporated cells can be cultured on-chip inside such microfluidic systems for further on-chip operations or long-term studies.

Finally, cells may be fused by using fusogenic proteins, such as those employed by viruses to enter or exit the cell. One exemplary fusogenic protein, derived from measles hemagglutinin (H) protein, is described in Nakamura et al., Nature Biotechnology, Vol. 22, p. 331 (2004). For example, a myeloma cell can be genetically engineered to express a fusogenic protein. The fusogenic protein may be further engineered to recognize immunoglobulin molecules on the surface of a B cell. The fusogenic protein may also be further engineered so that the expression of the fusogenic protein is shut off after the fusion event.

In certain embodiments, immortalized antibody-producing cells are further cultured, either within a module of a microfludic device or in a separate culture dish (such as microtiter wells), to remove unsuccessful fusion products and to allow for further proliferation of the immortalized antibody-producing cells. For example, hybridoma cells may be cultured and screening according to the traditional hybridoma protocols that are well known in the art (e.g., cultured in HAT medium in microtiter wells).

Alternatively, the microfluidic devices may have a cell expansion module, in which each of collected immortalized antibody-producing cells (e.g., hybridoma cells) are placed in a separate well, and cultured in appropriate selection medium (e.g., HAT medium). Alternatively, fusion cells may be sorted and transferred to a separate cell expansion device. See, e.g., U.S. Pat. No. 7,169,577, corporate herein by reference, for exemplary microfluidic cell expansion devices.

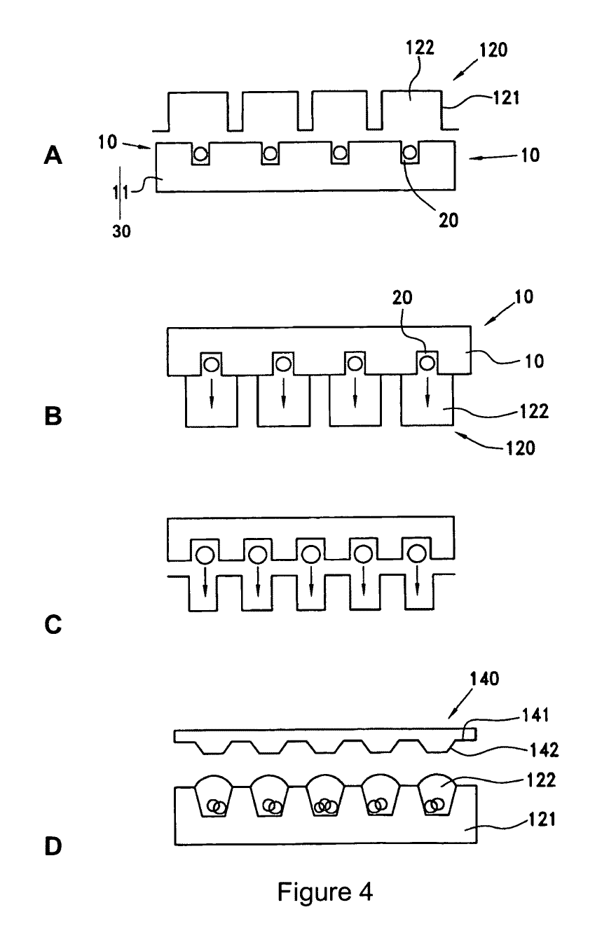

FIG. 4 provides an exemplary cell expansion devise. Hybridoma cells 30 are sorted and placed in a cell isolation region 20 of a cell isolation device 10. The cell expansion device 120 comprises a structure 121 defining a plurality of wells 122 with adjacent wells having the same pitch as the pitch defined by adjacent cell isolation regions of the cell isolation device 10. In particular, each well 122 in a test orientation of the cell expansion device 120, encompasses a cavity surrounded by two lateral sides connected to a bottom side. Preferably, each well is also characterized by having a greater volume than respective ones of the cell isolation region 20. “Wells” as used herein are specific to the cell expansion device, whereas cell isolation regions as used herein are specific to the cell isolation device. In general as seen in FIG. 4A, cell expansion device 120 is utilized by orienting structure 121 with housing 11 of cell isolation device 10 such that wells 122 overlie corresponding ones of the cell isolation regions 20.

Structure 121 is then placed in direct contact with housing 11 to form a seal such that fluid communication between cells 30 in adjacent cell isolation regions is inhibited. As seen in FIG. 4B, the mated cell isolation device/cell expansion device is then inverted and cells 30 are transferred, for example by centrifugal force, from cell isolation regions 20 to wells 122.

Wells 122 may be of any shape, but wells with circular or square shaped top plan view (or transverse cross-sections) are preferred as these shapes are commonly used in the industry. Notwithstanding the shape, wells 122 of expansion device 120 preferably have a greater volume than cell isolation regions 20 such as, for example, having a greater diameter (as illustrated in FIG. 4B) or a greater depth (as illustrated in FIG. 4C). In one embodiment, the lateral surfaces of well 122 are canted relative to one another or to the bottom surface of the well 122 in a test orientation of cell expansion device 120 as shown in FIG. 4D. Canted wells 122 permit easier access by allowing for lateral movement when performing downstream experiments on cells 30 in wells 122 and may create a more uniform interface with a small cell isolation region 20. On the other hand, a well 122 that has lateral sides that are at a 90° angle to the bottom side of the well 122 in a test orientation of the cell expansion device 120 provide a greater volume, which may increase the time available for the detection step to be described below.

Structure 121 is intended to define any number of wells 122, but preferably defines the number of wells in a standard microtiter plate such as a 24-, 96-, 384-, 768-, or 1536-well plate. More preferably, structure 121 defines the number of wells of a 384-well plate or 1536-well plate. Approximately 2 μl of liquid can be held per well 122 in a standard 1536 well plate. Larger well volumes can be achieved by extending the depth of the well 122 without compromising the correspondence between the pitch between adjacent wells 122 and the pitch between adjacent cell isolation regions 20.

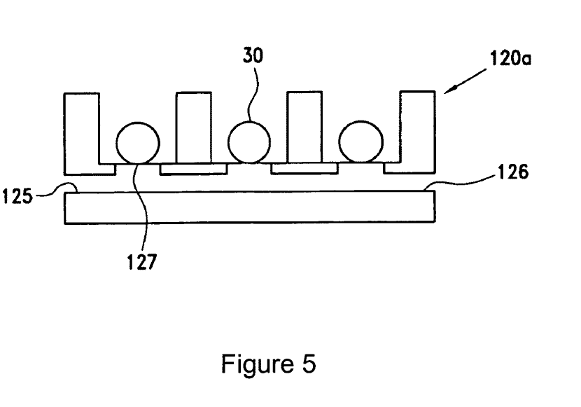

In another embodiment of cell expansion device 120 as seen in FIG. 5, cell expansion device 120a is configured such that the respective wells 122a are accessible from a bottom side 127 thereof in a test orientation of cell expansion device 120a. Structure 123a further defines an entrance port 125 and optionally, an exit port 126. The wells' 122a accessibility from the bottom side 127 permits exchange of media, thereby facilitating cell expansion and refeeding. Cells 30 are retained from passing through the access to the bottom side 127 of well 122a by a block such as a size constraint or semi-permeable membrane, such as a dialysis membrane, nitrocellulose or perforated polydimethylsialoxane, for example. The semi-permeable membrane of this embodiment also allows for gas exchange while reducing the evaporation of liquid contained in each well 122a. The initial flow-through of old medium may be drained by positive pressure, vacuum, or gravity to capture liquid potentially containing antibody or another product of the desired biological activity, on a surface designed for sampling/detection, e.g., nitrocellulose, glass, urethanes, rubber, molded plastic, or polydimethylsialoxane.

Preferably, the material used for the manufacture of cell expansion device 120 comprises any rigid or flexible material such as glass, urethanes, rubber, molded plastic, co-polymer or polymer, more preferably urethanes, rubber, molded plastic, polymethylmethacrylate (PMMA), polycarbonate, polytetrafluoroethylene (TEFLON™), polyvinylchloride (PVC), polydimethylsiloxane (PDMS), polysulfone, and the like, and most preferably PDMS. Such materials are readily manufactured from fabricated masters, using well known molding techniques, such as injection molding, embossing or stamping, or by polymerizing the polymeric precursor material within the mold. Such materials are preferred for their ease of manufacture, low cost and disposability, as well as their general inertness to most extreme reaction conditions.

Once the cells 30 are transferred from cell isolation device 10 to cell expansion device 122, the cells 30 are incubated in the cell expansion device 122, for a sufficient amount of time to allow for proliferation of cells 30. Cell expansion device 122 is intended to be used at any biologically viable temperature. Lowering the incubation temperature of the cells (i.e., from 37° C. to 18° C.) may, however, slow the metabolic processes of the cells 30 and reduce cell cloning time, thus extending the time for assay. Alternatively, media that is optimal for exhibiting the desired biological activity that is to be screened but not optimal for cell proliferation could also be used to extend the time for assay.

### 5. Detecting and Isolating Immortalized Immunoglobulin-Producing Cells

In another aspect, the invention further relates to detecting and isolating immortalized antibody-producing cells, such as hybridoma cells.

The immortalized antibody-producing cells can be screened using traditional techniques well known in the art. For example, immortalized antibody-producing cells, such as hybridoma cells, may be cultured in a suitable culture medium that preferably contains one or more substances that inhibit the growth or survival of the unfused, immortalized cells. Typically, if the parental cells lack the enzyme hypoxanthine guanine phosphoribosyl transferase (HGPRT or HPRT), the culture medium for the hybridomas will include HAT medium, which substances prevent the growth of HGPRT-deficient cells.

The culture medium (or the supernatant) in which the immortalized antibody-producing cells (e.g., hybridoma cells) are cultured can then be assayed for the presence of monoclonal antibodies directed against the antigen of interest. Preferably, the binding specificity of monoclonal antibodies produced by the immortalized antibody-producing cells (e.g., hybridoma cells) is determined by immunoprecipitation or by an in vitro binding assay, such as radioimmunoassay (MA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired immortalized antibody-producing cells (e.g., hybridoma cells) are identified, the clones may be subcloned by limiting dilution procedures and grown by standard methods. See, e.g., Goding, Monoclonal Antibodies: Principles and Practice, Academic Press, (1986) pp. 59-103. Suitable culture media for this purpose include, for example, Dulbecco's Modified Eagle's Medium and RPMI-1640 medium. Alternatively, the immortalized antibody-producing cells (e.g., hybridoma) cells may be grown in vivo as ascites in a mammal.

The monoclonal antibodies secreted by the subclones may be isolated or purified from the culture medium or ascites fluid by conventional immunoglobulin purification procedures such as, for example, PROTEIN A-SEPHAROSE™ (Pharmacia Biotech AB, Uppsala, Sweden), hydroxylapatite chromatography, gel electrophoresis, dialysis, or affinity chromatography.

In another aspect, the invention further relates to detecting and isolating immortalized antibody-producing cells, such as hybridoma cells, using a microfluidic device. In certain embodiments, immortalized immunoglobulin-producing cells, such as hybridoma cells, are further screened using an antigen, or its immunogenic portion thereof, attached to a detectable marker.

The invention further provides for the identification and, optionally, the sorting of intact microcapsules where this is enabled by the sorting techniques being employed. In certain embodiments, microcapsule identification and, optionally, sorting relies on a change in the optical properties of the microcapsule, for example absorption or emission characteristics thereof, for example alteration in the optical properties of the microcapsule resulting from a reaction leading to changes in absorbance, luminescence, phosphorescence or fluorescence associated with the microcapsule. All such properties are included in the term “optical”. In such a case, microcapsules can be identified and, optionally, sorted by luminescence, fluorescence or phosphorescence activated sorting.

In certain embodiment, cells secreting a desired immunoglobulin can be screened by detecting a change in the optical properties of the microcapsule. The change in optical properties of the microcapsule after binding of an antibody to an antigen may be induced in a variety of ways. For example, the antigen may be attached to a compound whose optical properties may be modified upon the binding of the antibody (for example, the fluorescence of the compound is quenched or enhanced upon antibody-binding).

Alternatively, a second microcapsule, comprising a secondary antibody attached to a detectable marker (e.g., a fluorescent or luminescent anti-mouse antibody) may be introduced by the microfluidic device. The second microcapsule can be coalesced with the first microcapsule using a microfluidic device, as described above.

In a preferred embodiment of the invention, the microcapsules will be analyzed and, optionally, sorted by flow cytometry. Many formats of microcapsule can be analyzed and/or sorted directly using flow cytometry.

A variety of optical properties can be used for analysis and to trigger sorting, including light scattering (Kerker, Cytometry, 1983 July; 4(1): 1-10) and fluorescence polarization (Rolland, J Immunol Methods, 1985 Jan. 21; 76(1):1-10).

In a preferred aspect of the invention, microcapsule identification relies on a change in the optical properties of the microcapsule resulting from antibody-antigen binding leading to luminescence, phosphorescence or fluorescence within the microcapsule.

In certain embodiments, the difference in optical properties of the microcapsules will be a difference in luminescence. For example, the secondary antibody can be attached to an enzyme, such as beta-galactosidase, alkaline phosphatase, or horseradish peroxidase, which subsequently converts a substrate into a luminescent product. As explained in detail above, because of encapsulation, the enzyme-based detection methods are highly sensitive. Substrate(s) of the enzyme can either be directly coencapsulated with the secondary antibody, coencapsulated with the antigen, or introduced as separate microcapsules, which are then fused with those antigen microcapsules as described above.

In certain embodiments, the difference in optical properties of the microcapsules will be a difference in fluorescence and, if required, the microcapsules will be sorted using a microfluidic or conventional fluorescence-activated cell sorter, or similar device, as described in detail above.

The methods of the current invention allow reagents to be mixed rapidly (in <2 ms), hence a spatially-resolved optical image of microcapsules in microfluidic network allows time resolved measurements of the reactions in each microcapsule. Microcapsules can, optionally, be separated using a microfluidic flow sorter to allow recovery and further analysis or manipulation of the molecules they contain. Advantageously, the flow sorter would be an electronic flow sorting device. Such a sorting device can be integrated directly on the microfluidic device, and can use electronic means to sort the microcapsules. Optical detection, also integrated directly on the microfluidic device, can be used to screen the microcapsules to trigger the sorting. Other means of control of the microcapsules, in addition to charge, can also be incorporated onto the microfluidic device.

The devices and methods of the invention also include other conventional embodiments wherein the cells are sorted. For example, as illustrated in FIG. 4D, after sufficient time has been allowed for cells 30 to grow and divide, a detection device 140 may be utilized to detect the antibody production of the cells. In one embodiment, as illustrated in FIG. 4D, detection device 140 comprises a surface 141 defining an array of prongs 142 upon which antigens are immobilized. In certain embodiments, surface 141 is a substantially planar surface. In one embodiment, prongs 142 are coated with the specific antigen(s) (or immunogenic portion thereof) used to immunize animals from which the cells being tested are derived. In use, detection device 140 is placed over structure 121 of cell expansion device 120 such that prongs 142 are in registration with wells 122. Prongs 142 are then immersed into wells 122 of cell expansion device 120 in order to allow antigens immobilized on prongs 142 to potentially bind to antibodies secreted/produced by cells, such as hybridoma cells in wells 120. Prongs 142 of detection device 140 are then removed from well 122 of cell expansion device 120 and then immersed into a solution of detectable secondary antibody. Secondary antibodies may be visualized by standard techniques known in the art such as by enzymatic color reactions or fluorescence. A prong surface may also be interfaced with mass spectrometer for additional assay flexibility. Other methods of labeling and detecting the antibodies are well within the knowledge of one of skill in the art and therefore are not discussed in detail herein.

### 6. Sequencing and Expressing a Desired Immunoglobulin

In another aspect, the invention further relates to identifying the sequence of a desired immunoglobulin. For example, after identifying a B cell that expresses a desired immunoglobulin from a pool of cells, the sequences the immunoglobulin's heavy (e.g., V_{H} region) and/or light (e.g., V_{L} region) chains can be obtained, e.g., by PCR or other techniques known in the art. Monoclonal antibodies can then be produced based on the heavy and light chain sequences, rendering the immortalization of the antibody-producing cells unnecessary.

In certain embodiments, one or more steps to identify the sequence of the immunoglobulin are performed under microfluidic control. For example, single-cell PCR may be used to amplify the mRNA(s) encoding the heavy and light chain sequences of a desired immunoglobulin. In some embodiments, single-cell PCR is performed by rupturing the cell without breaking the microcapsule. In other embodiments, the microcapsule can be broken before or during PCR.

The immunoglobulin may be sequenced using any suitable technique known to those of ordinary skill in the art. Examples of nucleic acid sequencing techniques include, but are not limited to, PCR, sequencing by synthesis techniques (e.g., using DNA synthesis by DNA polymerase to identify the bases present in the complementary DNA molecule), sequencing by ligation (e.g., using DNA ligases), sequencing by hybridization (using DNA microarrays), nanopore sequencing techniques, or the like. If necessary, the target nucleic acid sequence may be amplified, duplicated, or expanded by PCR, rolling circle replication or equivalent techniques.

In certain embodiments, PCR is used to identify the sequence of a desired immunoglobulin. For example, a PCR mixture may be divided between the aqueous droplets of a water/oil emulsion such that there is, in most cases, no more than one template DNA molecule per microcapsule. The emulsion then may be thermo-cycled and each of the template DNA molecules may be amplified in a separate microcapsule.

The nucleic acid sequence of a desired immunoglobulin may be inserted into a suitable host cell to recombinantly express the immunoglobulin. Examples of methods of transfecting a cell with a nucleotide sequence are well-known to those of ordinary skill in the art.

Alternatively, a desired immunoglobulin can be expressed without a host cell, e.g., in a cell-free expression system. Cell-free translation systems will often comprise a cell extract, typically from bacteria (see, e.g., Zubay, G. (1973) Annu. Rev. Genet., 7, 267-287; Zubay, G. Methods Enzymol., 65, 856-877; Lesley, S. A. (1991) J. Biol. Chem. 266, 2632-2638; Lesley, S. A. et al. (1995) Methods Mol. Biol. 37, 265-278), rabbit reticulocye (Pelham and Jackson, (1976), Eur. J. Biochem, 67, 247-256), wheat germ (Anderson, C W. et al. (1983) Methods Enzymol, 101, 635-644), etc., or are partially recombinant, cell-free, protein-synthesis systems reconstituted from elements of systems such as the Escherichia coli translation system (Shimizu, Y. et al. (2001) Nat. Biotechnol. 19, 751-755). Commercial cell-free translation systems are available from a number of suppliers including Invitrogen, Roche, Novagen, or Promega.

In some cases, the recombinantly expressed immunoglobulin may be further optimized, e.g., by directed evolution, and/or other modifications to obtain optimal activity or binding.

Immunoglobulins are typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2. Each light chain includes an N terminal variable (V) domain (V_{L}) and a constant (C) domain (C_{L}). Each heavy chain includes an N terminal V domain (V_{H}), three or four C domains (C_{H}s), and a hinge region collectively referred to as the constant region of the heavy chain. The C_{H} domain most proximal to V_{H} is designated as C_{H}1. The V_{H} and V_{L} domains consist of four regions of relatively conserved sequences called framework regions (FR1, FR2, FR3, and FR4), that form a scaffold for three regions of hypervariable sequences also referred to as complementarity determining regions CDRs. CDRs are referred to as CDR1, CDR2, and CDR3. CDR3 is typically the greatest source of molecular diversity within the antibody-binding site.

The immunoglobulins of the invention include complete 4-chain antibodies and antigen-binding fragments of complete antibodies. An antigen-binding fragment (also referred to as an antigen-binding portion) includes but is not limited to Fab, Fv and ScFv molecules. The Fab fragment (Fragment antigen-binding) consists of V_{H}-C_{H}1 and V_{L}-C_{L} domains covalently linked by a disulfide bond between the constant regions. The Fᵥ fragment is smaller and consists of V_{H} and V_{L} domains non-covalently linked. To overcome the tendency of non-covalently linked domains to dissociate, a single chain Fᵥ fragment (scFᵥ) can be constructed. The scFᵥ contains a flexible polypeptide that links (1) the C-terminus of V_{H} to the N-terminus of V_{L}, or (2) the C-terminus of V_{L} to the N-terminus of V_{H}. Repeating units of (Gly₄Ser)—often 3 or 4 repeats may be used as a linker, but other linkers are known in the art. See, Paul (1993) Fundamental Immunology, Raven Press, N.Y. for a more detailed description of other antibody fragments). While various antibody fragments are defined in terms of the digestion of an intact antibody, one of skill will appreciate that such fragments may be synthesized de novo either chemically, by utilizing recombinant DNA methodology, or by “phage display” methods. See, e.g., Vaughan et al. (1996) Nature Biotechnology, 14(3): 309-314, and PCT/US96/10287).

As specific non-limiting examples, the antibody may be murine, chimeric, humanized, human, etc.

### EXAMPLES

### Example 1

### Detection of Cell Surface IgG on Antigen-Specific Cells

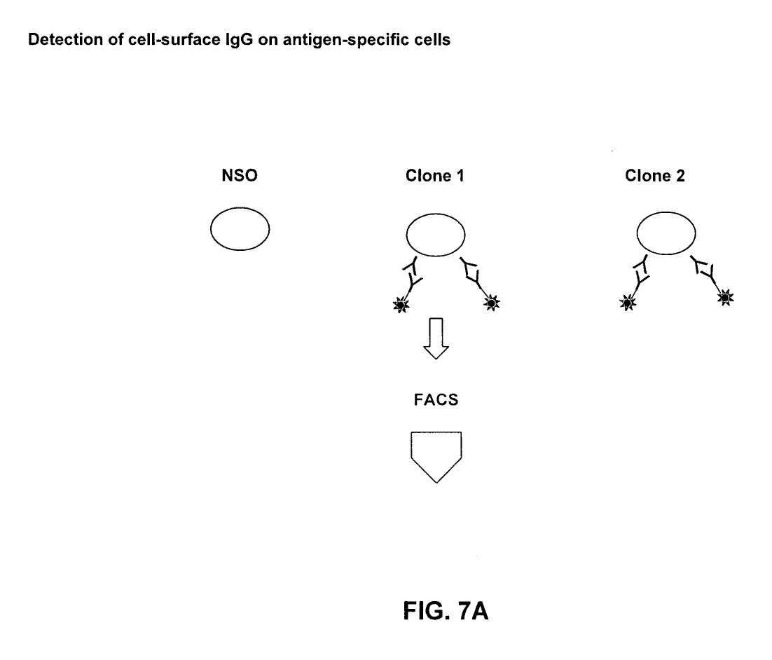

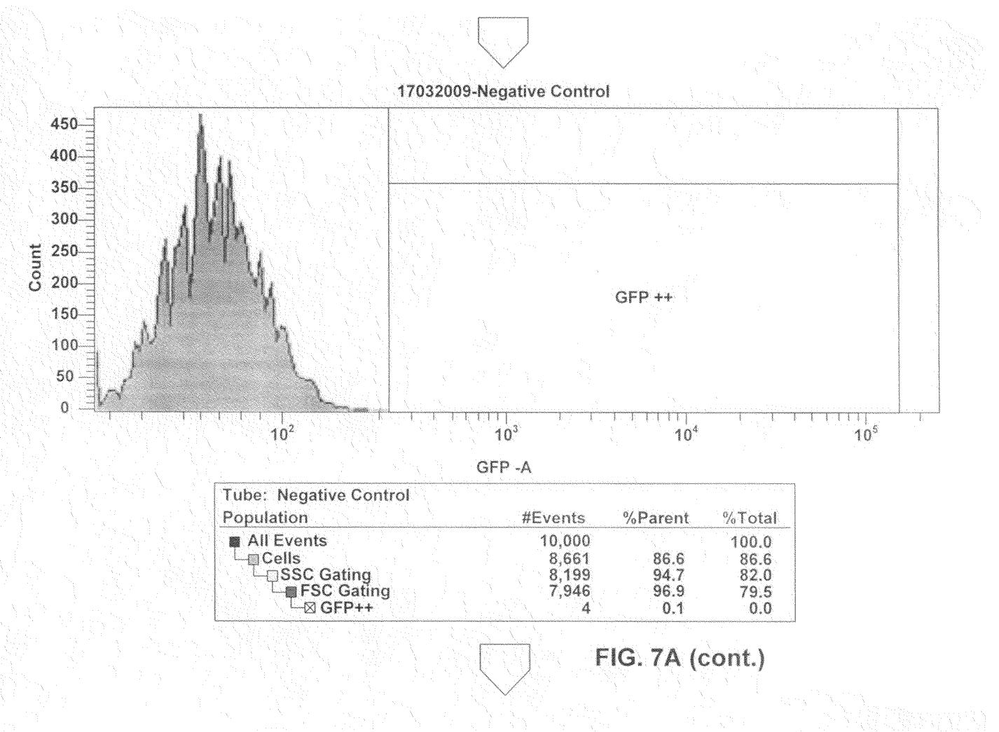

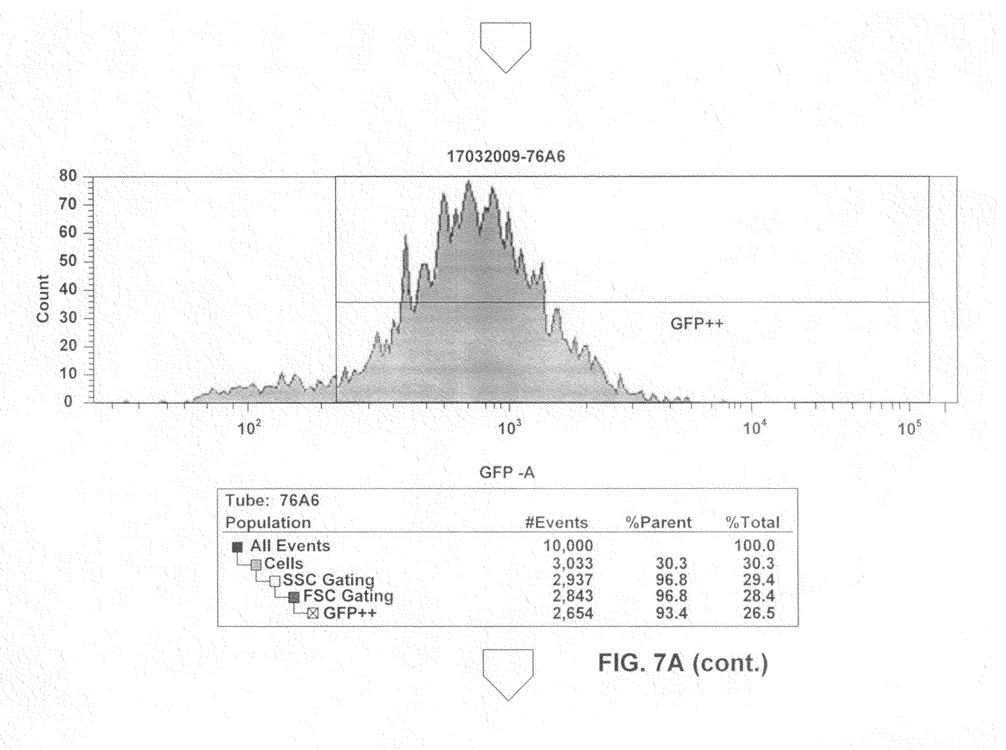

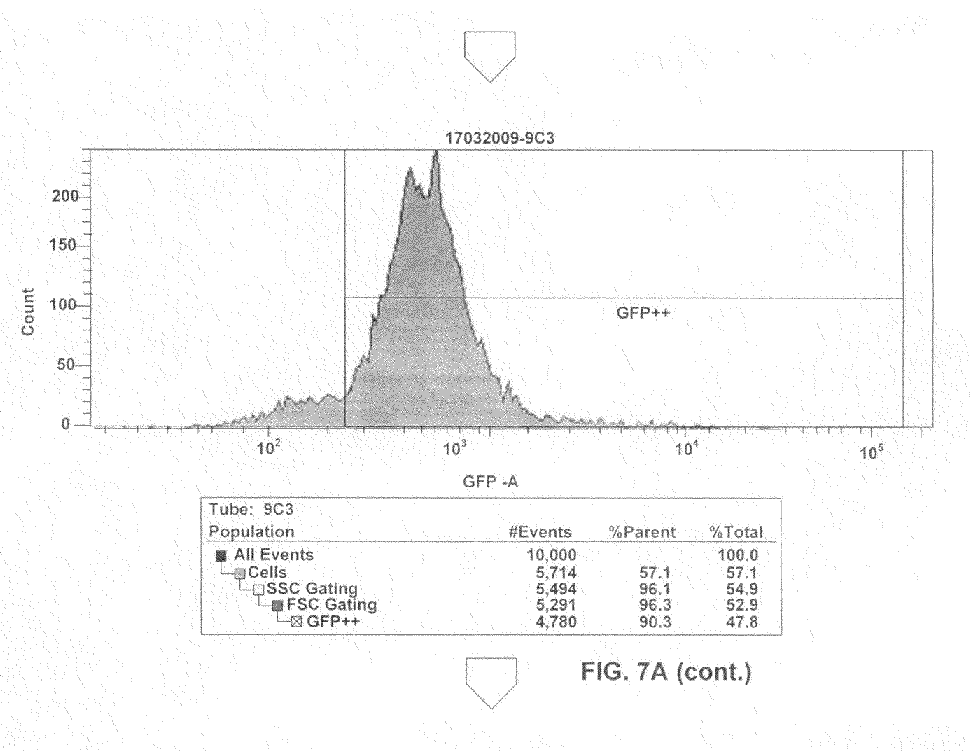

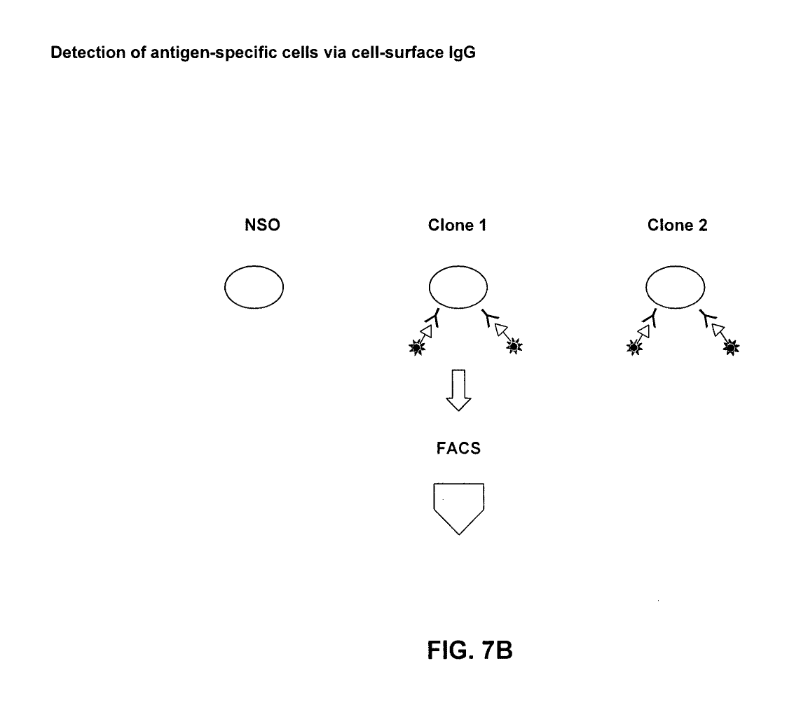

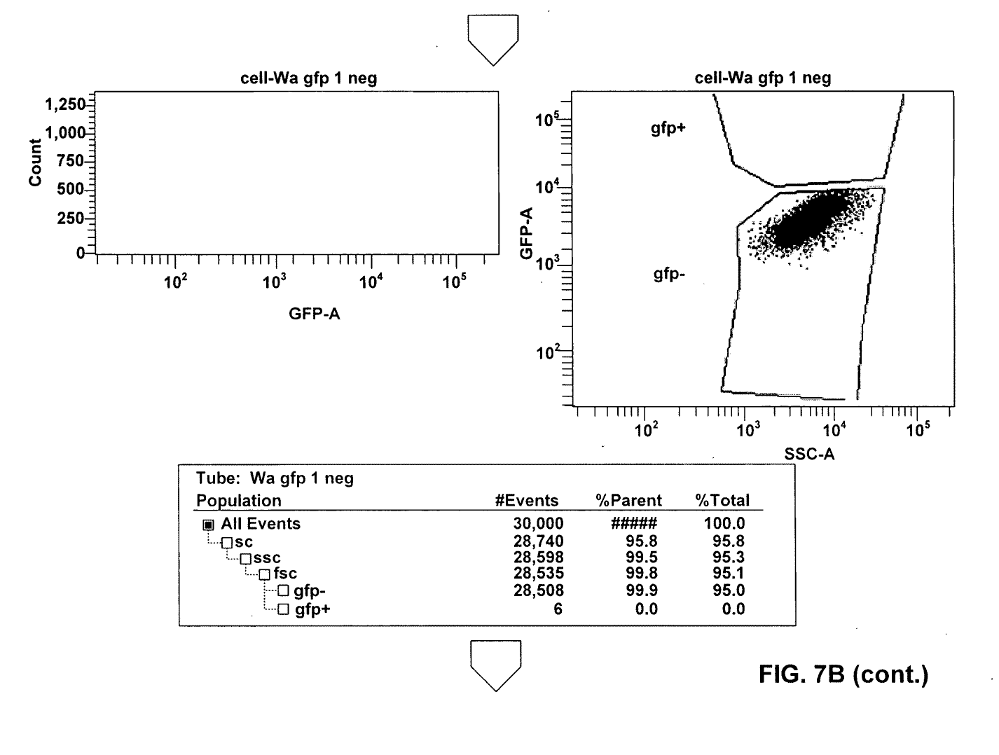

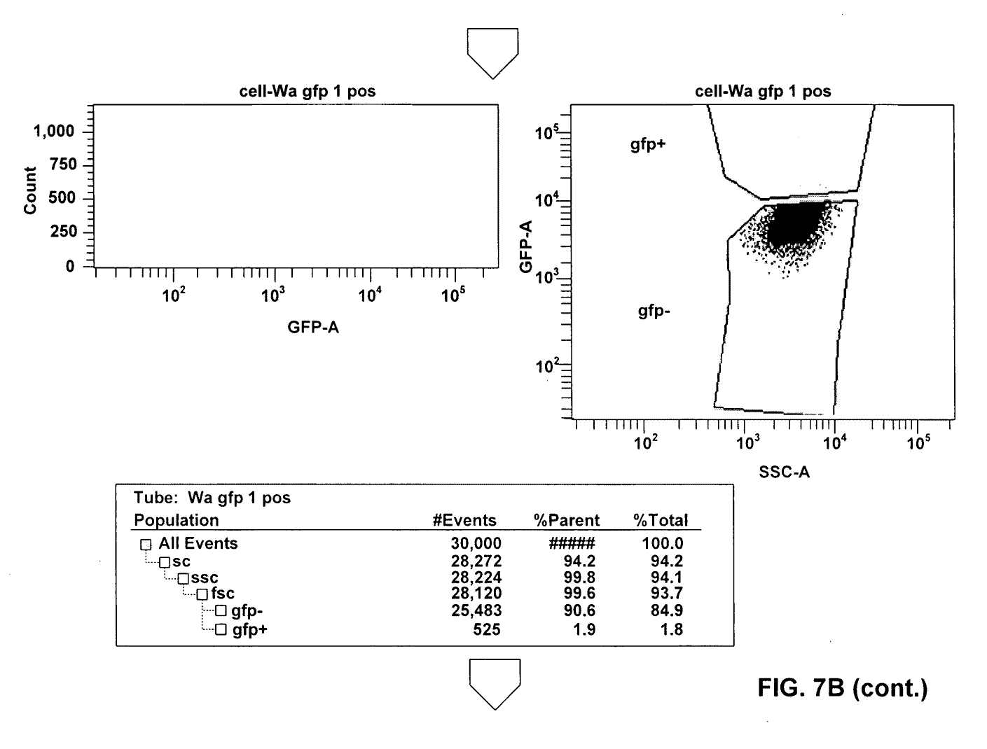

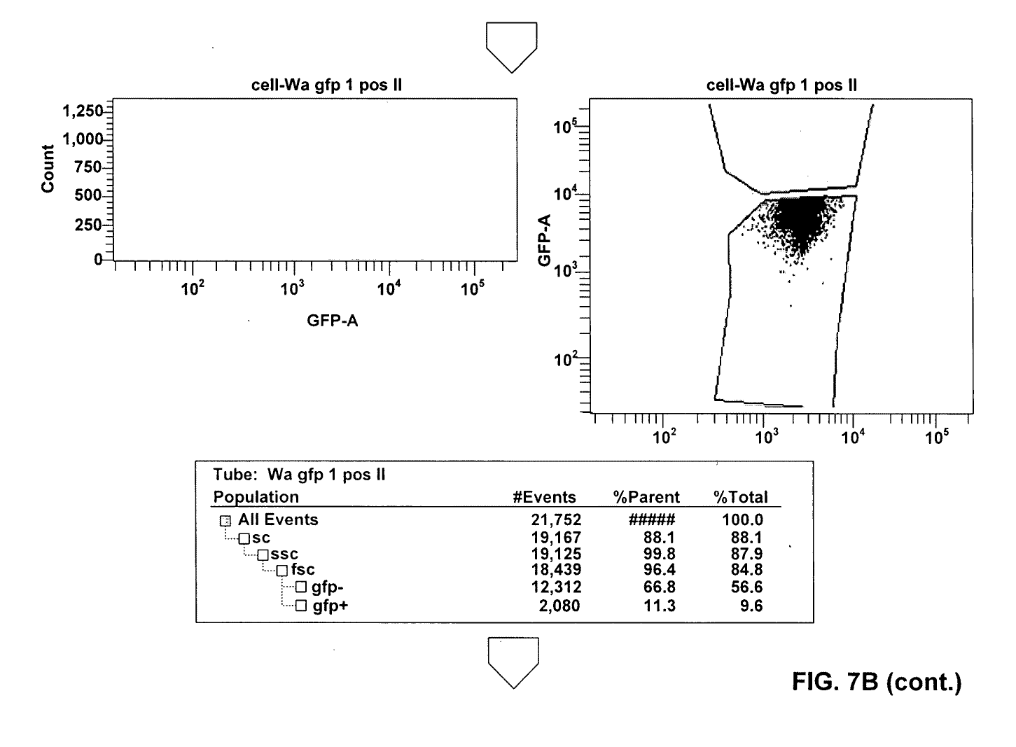

To detect cell surface IgG on antigen-specific B cells, we compared the expression of cell surface IgG on non-secreting “NSO” myeloma cells with that of hybridomas, which secret monoclonal antibodies against Ephrin B1 (cell lines 76A6 (“clone 1”) and 9C3 (“clone 2”)). These three cell lines were grown in DMEM supplemented with 10% fetal bovine serum and then washed three times with phosphate-buffered saline. Cells were then exposed to Alexa 488-conjugated goat anti-mouse IgG (Molecular Probes, Inc.), washed again with PBS, and analyzed by FACS (FIG. 7A). Significantly, the NSO cells, which secreted no detectable IgG, showed very low signals, with a mean of approximately 60 arbitrary units, in the “GFP” channel used to detect Alexa 488. In contrast, clones 76A6 and 9C3 showed surface IgG values of 800 and 400, respectively (FIG. 7A).

To determine whether these same hybridomas could be detected by direct binding of antigen, these cells were exposed to a fusion protein between the extracellular 150 amino acids of the ephrin B1 protein recognized by hybridomas 76A6 and 9C3 and the 10 amino acid cMyc epitope recognized by a rabbit polyclonal anti-cMyc rabbit antibody (Cell Signaling). Cells were then washed in PBS and incubated with the anti-Myc antibody followed by an Alexa 488-conjugated goat anti-rabbit IgG antibody preabsorbed against mouse immunoglobulins. Whereas the mean value for the NSO cells was 4000 in arbitrary units, the 76A6 and 9C3 lines showed values of 10,000 and 20,000 arbitrary units (FIG. 7B), indicating that we could readily detect antigen-positive cells compared to those that do not bind antigen. These data indicate that the hybridomas 76A6 and 9C3 retained the same dual splicing of immunoglobulins to effect both the secretion and membrane-association of the IgGs, and that presorting antigen-specific hybridomas or B cells from a pool of cells can potentially significantly enhance the efficiency of monoclonal antibody production.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

1. A method for producing an immortalized immunoglobulin-producing cell that expresses an immunoglobulin that bind to an antigen, comprising:
a) obtaining a plurality of B cells from a subject, wherein the subject that has been exposed to or immunized with the antigen, or an antigenic portion thereof;
b) detecting and selecting from said plurality of B cells one or more B cells that express the desired immunoglobulin as a cell-surface bound immunoglobulin;
c) fusing one or more cells of step b) with immortalized cells to produce one or more immortalized immunoglobulin-producing cells.

2. The method of claim 1, wherein step c) is performed under micro fluidic control.

3. The method of claim 1, wherein the immunoglobulin is IgG, IgM, or IgA.

4. The method of claim 1, wherein one or more B cells that express the desired immunoglobulin is detected and selected by the immunoglobulin's affinity to the antigen, or an antigenic portion thereof.

5. The method of claim 4, wherein the antigen, or an antigenic portion thereof, is attached to a microbead.

6. The method of claim 4, wherein the antigen, or an antigenic portion thereof, is attached to a detectable marker.

7. The method of claim 6, wherein the detectable marker is a fluorescent marker.

8. The method of claim 6, wherein the detectable marker is a luminescent marker.

9. The method of claim 6, wherein the antigen, or an antigenic portion thereof, is attached to an enzyme that generates luminescence.

10. The method of claim 9, wherein the enzyme is beta-galactosidase, alkaline phosphatase, or horseradish peroxidase.

11. The method of claim 1, wherein one or more B cells that express the desired immunoglobulin is detected and selected by FACS.

12. The method of claim 1, wherein step (b) further comprises:
1) compartmentalizing the plurality of B cells into microcapsules, such that only one B cell is present in any one microcapsule;
2) detecting and selecting one or more microcapsules in which the compartmentalized cells express the desired immunoglobulin;
wherein at least one of the steps 1) or 2) is performed under microfluidic control.

13. The method of claim 1, wherein the immortalize cells are myeloma cells.

14. The method of claim 1, wherein step c) further comprises:
1) compartmentalizing the B cells into microcapsules, such that only one B cell is present in any one microcapsule;
2) compartmentalizing the immortalized cells into microcapsules, such that only one immortalized cell is present in any one microcapsule; and
3) coalescing a B cell microcapsule formed in step 1) with a microcapsule formed step 2) under the influence of an electric field to cause the fusion of a B cell with an immortalized cell;
wherein at least one of the steps 1), 2), or 3) is performed under microfluidic control.

15. The method of claim 1, wherein the B cells and the immortalized cells are fused by electrofusion.

16. The method of claim 1, wherein the B cells and the immortalized cells are fused by a fusogenic protein.

17. The method of claim 1, further comprising culturing the immortalized immunoglobulin-producing cells to form a cell culture.

18. The method of claim 1, further comprising detecting the presence of the desired immunoglobulin expressed by the immortalized immunoglobulin-producing cell(s) of step (c).

19. The method of claim 18, further comprising:
1) compartmentalizing the immortalized immunoglobulin-producing cell(s) into microcapsules, such that only one immortalized immunoglobulin-producing cell is present in any one microcapsule; and
2) detecting the presence of the desired immunoglobulin expressed by the compartmentalized cells; and
wherein at least one of the steps 1) or 2) is performed under micro fluidic control.

20. The method of claim 18, wherein the presence of the desired immunoglobulin is determined by its affinity to the antigen, or an antigenic portion thereof.

21. The method of claim 20, wherein the immunoglobulin-antigen binding is detected by fluorescence.

22. The method of claim 20, wherein the immunoglobulin-antigen binding is detected by luminescence.

23. A method for screening a cell that expresses a desired immunoglobulin among a repertoire of cells, comprising:
a) compartmentalizing the repertoire of cells into microcapsules, such that only one cell is present in any one microcapsule; and
b) detecting the expression of the desired immunoglobulin by the cell;
wherein one of the steps a) orb) is performed under micro fluidic control.

24. A method for producing a fusion cell, comprising:
a) compartmentalizing a first population of cells into microcapsules, such that only one cell is present in any one microcapsule;
b) compartmentalizing a second population of cells into microcapsules, such that only one cell is present in any one microcapsule; and
c) coalescing a microcapsule formed in step a) with a microcapsule formed step b) under the influence of an electric field to cause the fusion of a cell from the first population with a cell from the second population;
wherein at least one of the steps a), b), or c) is performed under micro fluidic control.

25. A method for producing an immunoglobulin that bind to an antigen, comprising:
a) obtaining a plurality of B cells from a subject, wherein the subject that has been exposed to or immunized with the antigen, or an antigenic portion thereof;
b) detecting and selecting one or more B cells that express the desired immunoglobulin from the plurality of cells;
c) identifying the sequence the immunoglobulin's heavy and/or light chains from the selected B cell(s).

26. The method of claim 25, wherein one or more steps to identify the sequence of the immunoglobulin are performed under microfluidic control.

27. The method of claim 25, wherein the sequence the immunoglobulin is identified by PCR.

28. A method of identifying at least two B cells, each expressing an immunoglobulin that binds to an antigen, comprising:
a) obtaining a plurality of B cells from a subject, wherein the subject that has been exposed to or immunized with at least two different antigens, or their antigenic portion thereof;
b) attaching each antigen, or its antigenic portion thereof, to a unique detectable marker, such that each of the antigens is associated with a different detectable marker;
c) detecting and selecting two or more B cells that express the desired immunoglobulins from the plurality of cells, wherein the binding of an immunoglobulin to an antigen is determined by the unique detectable marker associated with the antigen.

29. The method of claim 28, further comprising: fusing the selected B cells of step c) with immortalized cells to produce immortalized immunoglobulin-producing cells.

30. The method of claim 28, further comprising: identifying the sequences the immunoglobulins heavy and/or light chains from the selected B cells of step c).

31. A method, comprising: providing a plurality of microfluidic droplets, wherein at least some of the microfluidic droplets contain non-immortalized cells; and determining a characteristic of an protein secreted by the non-immortal cells within the microfluidic droplets.

32. The method of claim 31, wherein the non-immortal cells are B-cells, and the protein is an immunoglobulin

33. A method, comprising: removing blood cells from a subject; encapsulating the blood cells in a plurality of microfluidic droplets; and at least partially separating, from the plurality of microfluidic droplets, microfluidic containing antibody-producing cells.

34. A method, comprising: providing a plurality of microfluidic droplets contained within a liquid, wherein at least some of the microfluidic droplets contain antibody-producing cells; and culturing the antibody-producing cells to secrete antibodies or portions thereof, wherein at least some of the antibody-producing cells are non-immortal cells.


SEQUENCE LISTING (1 sequence)

SEQ ID NO: 1 - protein, 52 aa, Homo Sapiens
WTTITIFITLFLLSVCYSATVTFFKVKWIFSSVVDLKQTIIPDYRNMIGQGA